(19) United States
(12) Patent Application Publication
(10) Pub. No.: US 2010/0178651 A1
(43) Pub. Date: Jul. 15, 2010

(54) Bifunctional Predictors of Cancer Treatment Sensitivity and Resistance

(76) Inventors: Christos Hatzis, Melrose, MA (US); Fraser W Symmans, Houston, TX (US); Lajos Pusztai, Pearland, TX (US)

Correspondence Address: Baker Botts L.L.P, 910 Louisiana Street, One Shell Plaza, HOUSTON, TX, 77002, US

(21) Appl. No.: 12/513,338

(22) PCT Filed: Nov. 5, 2007
(86) PCT No.: PCT/US07/83651

§ 371 Date: May 1, 2009

Related U.S. Application Data
(60) Provisional application No. 60/864,331, filed on Nov. 3, 2006.

(51) Int. Cl. C12Q 1/68; G01N 33/574

(52) U.S. Cl. 435/6; 435/7.23

(57) ABSTRACT

The present invention provides the identification of genes that are expressed in tumors to indicate responsiveness or resistance to an agent or class of agents. More particularly, these genes show expression associated with responsiveness to one agent or class of agents is inversely related to expression in association with another class of agents. One or more of theses genes of the present invention can be used as markers (or surrogate markers) to identify tumors that are likely to be successfully treated by the responsive agent as well as to identify tumors that should avoid treatment by the resistant agent.

### CROSS-REFERENCE TO RELATED APPLICATION

This application claims the benefit of U.S. Provisional Application Ser. No. 60/864,331 filed Nov. 3, 2006, which is incorporated by reference herein.

### BACKGROUND

The present invention relates generally to the field of molecular markers for predicting cancer outcomes such as response to drug treatments. More particularly, it relates to expression profiles of specific genes and their dual association with responsiveness and resistance to agents in cancer treatments.

Long term outcome of patients with newly diagnosed invasive breast cancer depends on the inherent biological aggressiveness of the disease and its sensitivity to adjuvant therapies. Patients with estrogen receptor (ER) positive breast cancer who are at substantial risk for recurrence often receive both adjuvant endocrine and chemotherapies. Prolonged recurrence free survival in these patients may be achieved from effective eradication of micrometastases by adjuvant endocrine treatment or by chemotherapy or by the combination of both. When novel predictive molecular markers of survival are proposed, it is imperative that they distinguish patients with efficacy of some form of systemic treatment from those who have inherently good prognosis in the absence of any adjuvant therapy.

Expression of genes such as microtubule associated protein (MAP)-Tau (referred to hererin as “Tau”) in breast cancer has been reported to be associated with higher rates of pathologic complete response (pCR) to chemotherapy (Rouzier, 2005). Tau expression correlates also closely with ER expression in human breast cancer (Frasor, 2004; Wang, 2005). This raises the possibility that certain gene sequences such as Tau may be a marker of estrogen activity as well as benefit from or resistance to chemotherapy in breast cancer. With the growing need to identify patients who benefit from use of specific agents or of combination therapies, markers that may simultaneously identify benefit from endocrine therapy and chemotherapy can offer considerable utility. We have identified several genes such as Tau that may have simultaneous correspondence with pCR or resistance to chemotherapy and with estrogen activity leading to sensitivity to endocrine treatment.

### SUMMARY

In the present invention, gene sequences have been identified as simultaneous markers of estrogen activity in breast cancer and, therefore as a predictor of benefit from anti-estrogen therapy, as well as a marker of benefit or resistance to chemotherapy. A bifunctional nature of expression of such gene sequences suggests an inverse relationship to benefit from treatment between an endocrine agent such as tamoxifen and from chemotreatment. Low expression of gene sequences such as microtubule associated protein Tau in breast cancer has been previously reported to be associated with higher rates of pathologic complete response (pCR) to chemotherapy with paclitaxel and 5-fluorouracil, doxorubicin, cyclophosphamide (T/FAC) (Rouzier, 2005). Expression of such gene sequences may also correlate closely with ER expression in human breast cancer. For example, Tau is included in several previously reported ER gene signatures (West, 2001; Symmans, 2005). Such genes, arising from imperfect estrogen response elements upstream to its promoter or as otherwise estrogen-induced sequences in cancer cells in vitro (Ferreira, 1991; Lew, 1993; Frasor, 2004), may provide effective markers for monitoring response to endocrine- and chemo-treatments simultaneously.

### DRAWINGS

FIG. 1 shows selection probabilities P_{g}(50), P_{g}(100), P_{g}(200) for the top-ranking probe sets in terms of their Spearman's rank correlation with the ESR1 transcript (probe set 205225_at) plotted as a function of the probe set's rank in the original dataset. Probabilities were estimated from 1000 bootstrap samples of the original dataset.

FIG. 2 shows the distribution of ranks of the top 200 genes estimated from 1000 bootstrap replications of the original dataset as a function of the magnitude of the Spearman's rank correlation with the ESR1 transcript.

FIGS. 3A-B describes the expression of MAP-tau (MAPT) in ER-negative and ER positive cancers from two different patient cohorts. Scales on the Y-axis are different due to different normalization procedures. (FIG. 3A) Patients who did not receive any systemic adjuvant therapy (n=286, surgical biopsy specimens) and (FIG. 3B) patients who received neoadjuvant T/FAC chemotherapy (n=133, fine needle aspiration specimens). The expression of MAP-tau mRNA (203929_s_at) was significantly higher in ER-positive cancers for both cohorts (Wilcoxon test p<0.0001 in both cases). Scales on the Y-axis are different due to different normalization procedures.

FIGS. 4 A-C shows a plot of rank ordered MAP-Tau mRNA expression levels for three distinct ER-positive patient cohorts. The plots indicate substantial variation in MAPT expression within ER-positive cancers. (FIG. 4A) patients who did not receive any systemic adjuvant therapy (n=209); (FIG. 4B) patients who received neoadjuvant T/FAC chemotherapy (n=82) and (FIG. 4C) patients who received adjuvant tamoxifen (n=267). The plots indicate substantial variation in MAPT expression within ER-positive cancers.

FIGS. 5 A-E describes MAP-Tau mRNA expression in ER-positive breast cancer and chemotherapy response and 5- and 10-year distant relapse. (FIGS. 5A-B) Lymph node negative patients without any systemic adjuvant therapy (n=209). (FIGS. 5C-D) Patients treated with 5 years of adjuvant tamoxifen (n=267). (FIG. 5E) Patients who received 6 months of preoperative T/FAC chemotherapy (pCR=pathologic complete response, RD=residual invasive cancer).

FIGS. 6 A-B are graphs of Kaplan-Meier distant relapse-free survival curves of ER positive patients by tertiles of Tau expression. (FIG. 6A) No systemic adjuvant therapy. (FIG. 6B) Tamoxifen treated patients. Patients are grouped by the tertiles of the distribution of MAPT mRNA expression (lowest 33%=tertile 1, highest 33%=tertile 3).

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The invention describes the use of one or more genes in predicting response or resistance simultaneously to chemotherapy and to endocrine treatment in a bifunctional manner, i.e. levels of the gene transcript, individually or in combination, are inversely predictive of potential high response in a given patient from chemotherapy and endocrine therapy.

The ability to choose an appropriate treatment at the outset can make the difference between cure and recurrence of a cancer, such as breast cancer. The present invention provides for the identification of patients who are the most likely to benefit from forms of systemic therapy, such as endocrine (hormonal) therapy and T/FAC chemotherapy, by assessing the differential expression of one or more of the responsiveness genes in a tumor sample from a patient. Such a predictive test can be used to select patients for pre- or postoperative treatment.

The expression level of a set or subset of identified responsiveness gene(s), or the proteins encoded by the responsive genes, may be used to: 1) determine if a tumor can be or is likely to be successfully treated by an agent or combination of agents; 2) determine if a tumor is responding to treatment with an agent or combination of agents; 3) select an appropriate agent or combination of agents for treating a tumor; 4) monitor the effectiveness of an ongoing treatment; and 5) identify new treatments (either single agent or combination of agents). In particular, the identified responsiveness genes may be utilized as markers (surrogate and/or direct) to determine appropriate therapy, to monitor clinical therapy and human trials of a drug being tested for efficacy, and to develop new agents and therapeutic combinations.

In certain embodiments, methods and compositions include genes (markers) that are expressed in cancer cells responsive or resistant to a given therapeutic agent and whose expression (either increased expression or decreased expression) correlates with responsiveness or resistance to a therapeutic agent. A “responsiveness gene” or “gene marker” as used herein is a gene whose increased expression or decreased expression is correlated with a cell's response to a particular therapy. Response may be described as a therapeutic response (sensitivity) and resistance as a lack of therapeutic response (residual disease, which may indicate resistance). Accordingly, one or more of the genes such as that in TABLE 1 can be used as markers (or surrogate markers) to identify tumors and tumor cells that are likely to be successfully treated by a therapeutic agent(s) or conversely, to identify tumors or tumor cells that are most likely to be resistant to a therapeutic agent(s).

Embodiments of the invention include methods for assessing the responsiveness or lack thereof of a tumor to therapy. In certain embodiments the methods comprise obtaining a sample of a tumor from a patient; evaluating the sample for expression of one or more markers such as that identified in Table 1; and assessing the responsiveness of the tumor to multiple forms of therapy based on the evaluation of marker expression in the sample. Marker refers to a gene or gene product (RNA or polypeptide) whose expression is related to response of a cancer to a therapy, either a positive (complete pathological response) or a negative response (residual disease). Expression of a marker may be assessed by detecting polynucleotides or polypeptides derived therefrom. In particular embodiments, the marker is the nucleic acid encoding one or more of the genes in Table 1 such as the microtubule-associated protein Tau or the encoded Tau polypeptide. In certain aspects, the tumor may be classified as sensitive when the therapy achieves an outcome of a complete pathological response or the gene expression profiles predicts that a tumor will have some probability of a complete pathological response.

TABLE 1
Description of gene sequence for predicting bifunctional response to therapy

| | | | | T-stat | |
|---|---|---|---|---|---|
| | | | | (MeanCR − | |
| No. | ProbeSet | LocusLink | Name | MeanNR)/se | P-val |
| 1 | 203928_x_at | 4137 | Microtubule-associated protein | −5.99 | 2.70 × 10⁻⁰⁷ |
| 2 | 203929_s_at | 4137 | Microtubule-associated protein | −5.52 | 1.26 × 10⁻⁰⁶ |
| 3 | 205074_at | 6584 | Solute carrier family 22 (organ | −5.13 | 5.45 × 10⁻⁰⁶ |
| 4 | 205696_s_at | 2674 | GDNF family receptor alpha 1 | −5 | 1.06 × 10⁻⁰⁵ |
| 5 | 219741_x_at | 79818 | Hypothetical protein FLJ21603 | −4.94 | 1.00 × 10⁻⁰⁵ |
| 6 | 218769_s_at | 57763 | “Ankyrin repeat, family A | −4.7 | 2.58 × 10⁻⁰⁵ |
| | | | (RFXAN” | | |
| 7 | 219981_x_at | 55044 | Hypothetical protein FLJ20813 | −4.66 | 4.44 × 10⁻⁰⁵ |
| 8 | 213234_at | 57613 | KIAA1467 protein | −4.6 | 3.73 × 10⁻⁰⁵ |
| 9 | 219197_s_at | 57758 | CEGP1 protein | −4.57 | 3.45 × 10⁻⁰⁵ |
| 10 | 201413_at | 3295 | Hydroxysteroid (17-beta) | −4.46 | 5.71 × 10⁻⁰⁵ |
| | | | dehydr | | |
| 11 | 209173_at | 10551 | Anterior gradient 2 homolog | −4.41 | 6.36 × 10⁻⁰⁵ |
| | | | (Xe | | |
| 12 | 214053_at | | Homo sapiens clone 23736 | −4.18 | 1.51 × 10⁻⁰⁴ |
| | | | mRNA s | | |
| 13 | 215304_at | | Human clone 23948 mRNA | −4.13 | 1.40 × 10⁻⁰⁴ |
| | | | sequence | | |
| 14 | 218692_at | 55638 | Hypothetical protein FLJ20366 | −4.13 | 1.76 × 10⁻⁰⁴ |
| 15 | 218976_at | 56521 | J domain containing protein 1 | −4.12 | 1.76 × 10⁻⁰⁴ |
| 16 | 212956_at | 23158 | KIAA0882 protein | −4.01 | 2.27 × 10⁻⁰⁴ |
| 17 | 217838_s_at | 51466 | RNB6 | −4.01 | 2.14 × 10⁻⁰⁴ |
| 18 | 218211_s_at | 79083 | Melanophilin | −3.95 | 3.05 × 10⁻⁰⁴ |
| 19 | 214164_x_at | 164 | Adaptor-related protein | −3.91 | 3.52 × 10⁻⁰⁴ |
| | | | complex | | |
| 20 | 204862_s_at | 4832 | “Non-metastatic cells 3, | −3.91 | 3.55 × 10⁻⁰⁴ |
| | | | protein” | | |
| 21 | 213527_s_at | 146542 | Similar to hypothetical protein | −3.85 | 4.33 × 10⁻⁰⁴ |

In certain embodiments, the therapy is selected from chemotherapy, and preferably T/FAC therapy, and an endocrine therapy. In still further embodiments, the therapy may be selected to chemotherapy alone, endocrine therapy alone, or a combination of chemotherapy and endocrine therapy.

In certain aspects of the invention, the tumor comprises breast cancer. In still other aspects the tumor is sampled by aspiration, biopsy, or surgical resection. Embodiments of the invention include assessing the expression of the one or more markers by detecting an mRNA species derived from one or more markers. In an embodiment, assessing the expression of one or more markers is by detecting a protein derived from a gene identified as a marker. A protein may be detected by immunohistochemistry, western blotting, or other known protein detection means. In a further embodiment, detection comprises microarray analysis, and more preferably the microarray is an Affymetrix Gene Chip. In other aspects of the invention, detection comprises nucleic acid amplification, preferably PCR. In still further aspects, detection is by in situ hybridization.

In still a further embodiment includes methods of monitoring a cancer patient receiving endocrine therapy. Methods of monitoring a cancer patient comprise obtaining a tumor sample from the patient during the therapy; evaluating expression of one or more markers such as that in TABLE 1 in the tumor sample; and assessing the cancer patient's responsiveness to chemotherapy, e.g., T/FAC therapy (taxane-paclitaxel/5-fluorouracil (5-FU), doxorubicine, and cyclophosphamide). A tumor sample may be obtained, evaluated and assessed repeatedly at various time points during chemotherapy.

In yet other embodiments include methods of assessing anti-cancer activity of a candidate substance. The methods comprise contacting a first cancer cell with a candidate substance; comparing expression of one or more markers such as that in TABLE 1 in a first cancer cell exposed to a candidate substance with expression of the markers in a second cancer cell not contacted with the candidate substance; and assessing the anti-cancer activity of the candidate substance. Anti-cancer activity can be the sensitization of a cancer cell to therapy, which may be evaluated by gene expression profiles. In certain aspects, the therapy is chemotherapy alone, preferably the chemotherapy is T/FAC therapy, an endocrine therapy, or a combination of chemotherapy and endocrine therapy.

Embodiments of the invention may also include kits for the determination of sensitivity or resistance of cancer comprising: (a) reagents for determining expression levels of one or marker genes in a sample; (b) algorithm and software for converting the expression levels of the said genes in a sample to determine the sensitivity of the patient to hormonal therapy or chemotherapy.

Low expression of Tau is associated with known clinicopathological predictors of response to chemotherapy such as ER-negative status and high nuclear grade. However, in contrast to these predictors that are not treatment regimen-specific, low Tau may predict extreme sensitivity to a particular drug, paclitaxel. Since Tau is a microtubule associated protein, Tau has a mechanistic role in determining cellular response to paclitaxel, which is a microtubule poison. The demonstration that down regulation of Tau by siRNA in breast cancer cells increases their sensitivity to paclitaxel but not to epirubicin suggests a direct role for Tau in determining response to this drug.

Although Tau in breast cancer is associated with higher rates of pathologic complete response (pCR) to chemotherapy, its expression also correlates closely with ER expression in human breast cancer. Tau is included in several previously reported ER gene signatures (Frasor, 2004; Wang, 2005). Furthermore, this gene contains an imperfect estrogen response element upstream to its promoter and it is an estrogen-induced gene in neurons and neuroblastoma cells, as well as in MCF-7 cells in vitro (Sotiriou, 2006; Loi, 2006; Hess, 2006). This raises the possibility that Tau expression is a marker of estrogen activity in breast cancer and therefore may predict benefit from anti-estrogen therapy.

It is contemplated that any method or composition described herein can be implemented with respect to any other method or composition described herein. Other objects, features and advantages of the present invention will become apparent from the examples. It should be understood, however, that the description and the specific examples, while indicating specific embodiments of the invention, are given by way of illustration only, since various changes and modifications within the spirit and scope of the invention will become apparent to those skilled in the art from this detailed description.

The expression level of a set or subset of identified responsiveness gene(s), or the proteins encoded by the responsive genes, may be used to: 1) determine if a tumor can be or is likely to be successfully treated by an agent or combination of agents; 2) determine if a tumor is responding to or resistant to treatment with an agent or combination of agents; 3) select an appropriate agent or combination of agents for treating a tumor; 4) monitor the effectiveness of an ongoing treatment; and 5) identify new treatments (either single agent or combination of agents). In particular, the identified responsiveness genes may be utilized as markers (surrogate and/or direct) to determine appropriate therapy, to monitor clinical therapy and human trials of a drug being tested for efficacy, and to develop new agents and therapeutic combinations.

In certain embodiments, methods and compositions include genes (markers) that are expressed in cancer cells responsive to a given therapeutic agent and whose expression (either increased expression or decreased expression) correlates with responsiveness to a therapeutic agent, see Table 1. A “responsiveness gene” or “gene marker” as used herein is a gene whose increased expression or decreased expression is correlated with a cell's response to a particular therapy. A response may be either a therapeutic response (sensitivity) or a lack of therapeutic response (residual disease, which may indicate resistance). Accordingly, one or more of the genes of the present invention can be used as markers (or surrogate markers) to identify tumors and tumor cells that are likely to be successfully treated by a therapeutic agent(s). In addition, the markers of the present invention can be used to identify cancers that have become or are at risk of becoming refractory to a treatment. Aspects of the invention include marker sets that can identify patients that are likely to respond or not to respond to a therapy.

In still further embodiments, the invention is directed to methods of treating or sensitizing a tumor in an individual to chemotherapy. These methods may comprise the steps of administering to the individual an agent that reduces the level of a gene whose down regulation is associated with pCR, e.g., Tau; thus sensitizing the tumor to chemotherapeutic agent such as paclitaxel; and administering an effective amount of a chemotherapeutic agent, such as paclitaxel. This method would be generally used to treat tumors which are resistant to chemotherapy, including breast tumors, glioblastomas, medulloblastomas, pancreatic adenocarcinomas, lung carcinomas, melanomas, and the like.

As used herein, cancer cells, including tumor cells, are “responsive” to a therapeutic agent if their rate of growth is inhibited or the tumor cells die as a result of contact with the therapeutic agent, compared to its growth in the absence of contact with the therapeutic agent. The quality of being responsive to a therapeutic agent is a variable one, with different tumors exhibiting different levels of “responsiveness” to a given therapeutic agent, under different conditions. In one embodiment of the invention, tumors may be predisposed to responsiveness to an agent if one or more of the corresponding responsiveness markers are expressed. Cancer, including tumor cells, is “non-responsive” to a therapeutic agent if its rate of growth is not inhibited (or inhibited to a very low degree) or cell death is not induced as a result of contact with the therapeutic agent, compared to its growth in the absence of contact with the therapeutic agent. The quality of being non-responsive to a therapeutic agent is a highly variable one, with different tumors exhibiting different levels of “non-responsiveness” to a given therapeutic agent, under different conditions.

As used herein, cancers, including tumor cells, refer to neoplastic or hyperplastic cells. Cancers include, but is not limited to, carcinomas, such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; leukemias and lymphomas such as granulocytic leukemia, monocytic leukemia, lymphocytic leukemia, malignant lymphoma, plasmocytoma, reticulum cell sarcoma, or Hodgkins disease; and tumors of the nervous system including glioma, meningoma, medulloblastoma, schwannoma or epidymoma.

In certain embodiments, all genes listed in Table 1 are identified that are differentially expressed between cancer cells sensitive to chemotherapy and those that are less sensitive or resistant. These responsiveness genes were identified by comprehensive gene expression profiling on samples that included fine needle aspiration specimens from human breast cancers obtained at the time of diagnosis. The set of or subsets of the genes may be used to assess the responsiveness of a cancer cell or tumor to a therapy. In certain embodiments, the set or a subset of responsiveness genes, in combination with a prediction algorithm, can be used to identify patients who have a better than average probability to experience a pathologic complete response (pCR) to a therapy, preferably chemotherapy, and more preferably T/FAC therapy and better than average probability to resistance to the therapy.

The present invention provides methods for determining whether a cancer is likely to be sensitive or resistant to a particular therapy or regimen. Although microarray analysis determines the expression levels of thousands of genes in a sample, only a subset of these genes are significantly differentially expressed between cells having different outcomes to therapy. Identifying which of these differentially expressed genes can be used to predict a clinical outcome requires additional analysis.

The genes described in the present invention are genes whose expression varies by a predetermined amount between tumors that are sensitive to chemotherapy, e.g., T/FAC, versus those that are not responsive or less responsive to a chemotherapy regimen. The following provides detailed descriptions of the genes of interest in the present invention. It is noted that homologs and polymorphic variants of the genes are also contemplated. As described herein, the relative expression of these genes may be measured through nucleic acid hybridization, e.g., microarray analysis. However, other methods of determining expression of the genes are also contemplated. It is also noted that probes for the following genes may be designed using any appropriate fragment of the full lengths of the nucleic acids sequences set forth in Table 1.

Gene expression data may be gathered in any way that is available to one of skill in the art. Typically, gene expression data is obtained by employing an array of probes that hybridize to several, and even thousands or more different transcripts. Such arrays are often classified as microarrays or macroarrays depending on the size of each position on the array.

In one embodiment, the present invention provides methods wherein nucleic acid probes are immobilized on a solid support in an organized array. Oligonucleotides can be bound to a support by a variety of processes, including lithography. It is common in the art to refer to such an array as a “chip.”

In one embodiment, gene expression is assessed by (1) providing a pool of target nucleic acids derived from one or more target genes; (2) hybridizing the nucleic acid sample to an array of probes (including control probes); and (3) detecting nucleic acid hybridization and assessing a relative expression (transcription) level.

Low expression of microtubule-associated protein Tau within the tumor at the time of diagnosis was significantly associated with complete pathologic response. The inventors have validated this association at the protein level on an independent set of patients (n=122) using immunohistochemistry. Low Tau expression was shown to be not only a marker of response but it causes sensitivity to paclitaxel in vitro. Down regulation or reduction in the expression of Tau with, for example, siRNA in cancer cells increases sensitivity to paclitaxel, but not to epirubicin. Tau partially protects cells from paclitaxel induced apoptosis by reducing paclitaxel binding to tubulin and reducing paclitaxel induced microtubule polymerization. These observations suggest that Tau is a clinically useful predictor of benefit from paclitaxel-containing adjuvant chemotherapy for breast cancer and that inhibition of Tau function sensitizes cells to paclitaxel. As described herein, low levels of Tau mRNA expression as measured by, but not limited to, cDNA microarrays or Tau protein expression detected by immunohistochemistry, are associated with higher rates of pathologic CR to T/FAC pre-operative chemotherapy for stage I-III breast cancer. This association was observed in two independent patient cohorts treated with essentially identical chemotherapy regimens. Pathologic CR in this context means complete eradication of the invasive cancer from the breast and lymph nodes by chemotherapy and has consistently been associated with excellent long-term survival that is independent of other tumor characteristics. The results indicate that assessment of Tau expression helps to identify patients at the time of diagnosis who have highly T/FAC sensitive tumors and therefore should receive this regimen if adjuvant or neoadjuvant chemotherapy is indicated.

Gene Expression: Providing a Nucleic Acid Sample

One of skill in the art will appreciate that in order to assess the transcription level (and thereby the expression level) of a gene or genes, it is desirable to provide a nucleic acid sample derived from the mRNA transcript(s). As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from the cDNA, a DNA amplified from the cDNA, an RNA transcribed from the amplified DNA, and the like, are all derived from the mRNA transcript. Detection of such derived products is indicative of the presence and abundance of the original transcript in a sample. Thus, suitable samples include, but are not limited to, mRNA transcripts of the gene or genes, cDNA reverse transcribed from the mRNA, cRNA transcribed from the cDNA, and the like.

Where it is desired to quantify the transcription level of one or more genes in a sample, the concentration of the mRNA transcript(s) of the gene or genes is proportional to the transcription level of that gene. Similarly, it is preferred that the hybridization signal intensity be proportional to the amount of hybridized nucleic acid. As described herein, controls can be run to correct for variations introduced in sample preparation and hybridization.

In one embodiment, a nucleic acid sample is the total mRNA isolated from a biological sample. The term “biological sample,” as used herein, refers to a sample obtained from an organism or from components (e.g., cells) of an organism, including diseased tissue such as a tumor, a neoplasia or a hyperplasia. The sample may be of any biological tissue or fluid. Frequently the sample will be a “clinical sample,” which is a sample derived from a patient. Such samples include, but are not limited to, blood, blood cells (e.g., white cells), tissue biopsy or fine needle aspiration biopsy samples, urine, peritoneal fluid, and pleural fluid, or cells therefrom. Biological samples may also include sections of tissues such as frozen sections taken for histological purposes.

The nucleic acid may be isolated from the sample according to any of a number of methods well known to those of skill in the art. One of skill in the art will appreciate that where expression levels of a gene or genes are to be detected, preferably RNA (mRNA) is isolated. Methods of isolating total mRNA are well known to those of skill in the art. For example, methods of isolation and purification of nucleic acids are described in Chapter 3 of Laboratory Techniques in Biochemistry and Molecular Biology (1993); Sambrook et al. (2001); Current Protocols in Molecular Biology (1987), all of which are incorporated herein by reference. Filter based methods for the isolation of mRNA are also known in the art. Examples of commercially available filter-based RNA isolation systems include RNAqueous® (Ambion) and RNeasy (Qiagen).

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

Methods of “quantitative” amplification are well known to those of skill in the art. For example, quantitative PCR involves simultaneously co-amplifying a known quantity of a control sequence. This provides an internal standard that may be used to calibrate the PCR reaction. The array may then include probes specific to the internal standard for quantification of the amplified nucleic acid.

Other suitable amplification methods include, but are not limited to polymerase chain reaction (PCR) (Innis, et al., 1990), ligase chain reaction (LCR) (see Wu and Wallace, 1989); Landegren, et al., 1988; Barringer, et al., 1990, transcription amplification (Kwoh, et al., 1989), and self-sustained sequence replication (Guatelli, et al., 1990).

In a particular embodiment, the sample mRNA is reverse transcribed with a reverse transcriptase, such as SuperScript II (Invitrogen), and a primer consisting of an oligo-dT and a sequence encoding the phage T7 promoter to generate first-strand cDNA. A second-strand DNA is polymerized in the presence of a DNA polymerase, DNA ligase, and RNase H. The resulting double-stranded cDNA may be blunt-ended using T4 DNA polymerase and purified by phenol/chloroform extraction. The double-stranded cDNA is then transcribed into cRNA. Methods for the in vitro transcription of RNA are known in the art and describe in, for example, Van Gelder, et al. (1990) and U.S. Pat. Nos. 5,545,522; 5,716,785; and 5,891,636, all of which are incorporated herein by reference.

If desired, a label may be incorporated into the cRNA when it is transcribed. Those of skill in the art are familiar with methods for labeling nucleic acids. For example, the cRNA may be transcribed in the presence of biotin-ribonucleotides. The BioArray High Yield RNA Transcript Labeling Kit (Enzo Diagnostics) is a commercially available kit for biotinylating cRNA.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense, as the target nucleic acids include both sense and antisense strands.

Labeling Nucleic Acids

To detect hybridization, it is advantageous to employ nucleic acids in combination with an appropriate detection means. Recognition moieties incorporated into primers, incorporated into the amplified product during amplification, or attached to probes are useful in the identification of nucleic acid molecules. A number of different labels may be used for this purpose including, but not limited to, fluorophores, chromophores, radiophores, enzymatic tags, antibodies, chemiluminescence, electroluminescence, and affinity labels. One of skill in the art will recognize that these and other labels can be used with success in this invention.

Examples of affinity labels include, but are not limited to the following: an antibody, an antibody fragment, a receptor protein, a hormone, biotin, Dinitrophenyl (DNP), or any polypeptide/protein molecule that binds to an affinity label.

Examples of enzyme tags include enzymes such as urease, alkaline phosphatase or peroxidase to mention a few. Colorimetric indicator substrates can be employed to provide a detection means visible to the human eye or spectrophotometrically, to identify specific hybridization with complementary nucleic acid-containing samples.

Examples of fluorophores include, but are not limited to, Alexa 350, Alexa 430, AMCA, BODIPY 630/650, BODIPY 650/665, BODIPY-FL, BODIPY-R6G, BODIPY-TMR, BODIPYTRX, Cascade Blue, Cy2, Cy3, Cy5, 6-FAM, Fluoroscein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, and Texas Red.

As mentioned above, a label may be incorporated into nucleic acid, e.g., cRNA, when it is transcribed. For example, the cRNA may be transcribed in the presence of biotinribonucleotides. The BioArray High Yield RNA Transcript Labeling Kit (Enzo Diagnostics) is a commercially available kit for biotinylating cRNA.

Means of detecting such labels are well known to those of skill in the art. For example, radiolabels may be detected using photographic film or scintillation counters. In other examples, fluorescent markers may be detected using a photodetector to detect emitted light. In still further examples, enzymatic labels are detected by providing the enzyme with a substrate and detecting the reaction product produced by the action of the enzyme on the substrate, and colorimetric labels are detected by simply visualizing the colored label.

So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin-bearing hybrid duplexes providing a label that is easily detected. For a detailed review of methods of labeling nucleic acids and detecting labeled hybridized nucleic acids see Laboratory Techniques in Biochemistry and Molecular Biology (1993).

Hybridization

As used herein, “hybridization,” “hybridizes,” or “capable of hybridizing” is understood to mean the forming of a double or triple stranded molecule or a molecule with partial double or triple stranded nature. The term “anneal” as used herein is synonymous with “hybridize.” The term “hybridization,” “hybridizes,” or “capable of hybridizing” are related to the term “stringent conditions” or “high stringency” and the terms “low stringency” or “low stringency conditions.”

As used herein “stringent conditions” or “high stringency” are those conditions that allow hybridization between or within one or more nucleic acid strands containing complementary sequences, but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as an mRNA or a nucleic acid segment thereof, or detecting at least one specific mRNA transcript or a nucleic acid segment thereof.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acids, the length and nucleobase content of the target sequences, the charge composition of the nucleic acids, and the presence or concentration of formamide, tetramethylammonium chloride or other solvents in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

The hybridization conditions selected will depend on the particular circumstances (depending, for example, on the G+C content, type of target nucleic acid, source of nucleic acid, and size of hybridization probe). Optimization of hybridization conditions for the particular application of interest is well known to those of skill in the art. Representative solid phase hybridization methods are disclosed in U.S. Pat. Nos. 5,843,663, 5,900,481, and 5,919,626. Other methods of hybridization that may be used in the practice of the present invention are disclosed in U.S. Pat. Nos. 5,849,481, 5,849,486, and 5,851,772.

DNA Chips and Microarrays

DNA arrays and gene chip technology provide a means of rapidly screening a large number of nucleic acid samples for their ability to hybridize to a variety of single stranded DNA probes immobilized on a solid substrate. These techniques involve quantitative methods for analyzing large numbers of genes rapidly and accurately. The technology capitalizes on the complementary binding properties of single stranded DNA to screen nucleic acid samples by hybridization (Pease et al., 1994; Fodor et al., 1991). Basically, a DNA array or gene chip consists of a solid substrate upon which an array of single stranded DNA molecules have been attached. For screening, the chip or array is contacted with a single stranded nucleic acid sample (e.g., cRNA), which is allowed to hybridize under stringent conditions. The chip or array is then scanned to determine which probes have hybridized.

The ability to directly synthesize on or attach polynucleotide probes to solid substrates is well known in the art. See U.S. Pat. Nos. 5,837,832 and 5,837,860, both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of the polystyrene or glass solid phases with poly-L-Lys or poly L-Lys, Phe, followed by the covalent attachment of either amino- or sulfhythyl-modified oligonucleotides using bi-functional crosslinking reagents (Running et al., 1990; Newton et al., 1993). When immobilized onto a substrate, the probes are stabilized and therefore may be used repeatedly.

In general terms, hybridization is performed on an immobilized nucleic acid target or a probe molecule that is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules).

The Affymetrix GeneChip system may be used for hybridization and scanning of the probe arrays. In a preferred embodiment, the Affymetrix U133A array is used in conjunction with Microarray Suite 5.0 for data acquisition and preliminary analysis.

Normalization Controls

Normalization controls are oligonucleotide probes that are complementary to labeled reference oligonucleotides that are added to the nucleic acid sample. The signals obtained from the normalization controls after hybridization provide a control for variations in hybridization conditions, label intensity, “reading” efficiency and other factors that may cause the hybridization signal to vary between arrays. For example, signals read from all other probes in the array can be divided by the signal from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few normalization probes are used and they are selected such that they hybridize well (i.e. no secondary structure) and do not match any target-specific probes. Normalization probes can be localized at any position in the array or at multiple positions throughout the array to control for spatial variation in hybridization efficiently.

In a particular embodiment, a standard probe cocktail supplied by Affymetrix is added to the hybridization to control for hybridization efficiency when using Affymetrix Gene Chip arrays.

Expression Level Controls

Expression level controls are probes that hybridize specifically with constitutively expressed genes in the sample. The expression level controls can be used to evaluate the efficiency of cRNA preparation.

Virtually any constitutively expressed gene provides a suitable target for expression level controls. Typically expression level control probes have sequences complementary to subsequences of constitutively expressed “housekeeping genes.”

In one embodiment, the ratio of the signal obtained for a 3′ expression level control probe and a 5′ expression level control probe that specifically hybridize to a particular housekeeping gene is used as an indicator of the efficiency of cRNA preparation. A ratio of 1-3 indicates an acceptable preparation.

Isolated Nucleic Acids for Analysis or Therapy

Nucleic acids of the present may be utilized in the preparation of therapeutic compositions. Certain genes related to the sensitivity of a cell to therapy that are expressed in a cell sensitive to therapy may be used therapeutically by increasing the expression of this gene or activity of an encoded protein in a cancer cell. Other genes related to resistance of a cell to a therapy may be down regulated transcriptionally or inhibited at the protein level by various therapies, such as anti-sense nucleic acid methods or small molecules. The protein products of these genes may also be targets for small molecules and the like, to either increase activity of a sensitizing protein or decrease activity of a resistance protein. Therapeutics that target the transcription of a gene, translation of RNA, and/or activity of an encoded protein may be used to sensitize cells to therapy, or in other aspects, may be used as a primary therapeutic apart from or in combinations with other therapies.

Nucleic acids of the present invention include nucleic acid isolated from a sample, probes, or expression vectors for both analysis of tumor responsiveness to therapy and cancer therapy. Certain embodiments of the present invention include the evaluation of the expression of one or more nucleic acids of the genes in Table 1. In certain embodiments, wild-type, variants, or both wild-type and variants of these sequences are employed. In particular aspects, a nucleic acid encodes for or comprises a transcribed nucleic acid. In other aspects, a nucleic acid comprises a nucleic acid segment of one or more of the genes, or a biologically functional equivalent thereof.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). “Nucleic acid” encompass the terms “oligonucleotide” and “polynucleotide,” each as a subgenus of the term “nucleic acid.” The term “oligonucleotide” refers to a molecule of between about 8 and about 100 nucleobases in length. The term “polynucleotide” refers to at least one molecule of greater than about 100 nucleobases in length.

In, certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. The term “gene” includes both genomic sequences, RNA or cDNA sequences or smaller engineered nucleic acid segments, including nontranscribed nucleic acid segments, including but not limited to the non-transcribed promoter or enhancer regions of a gene. Smaller engineered nucleic acid segments may encode proteins, polypeptides, peptides, fusion proteins, mutants and the like.

A polynucleotide of the invention may form an “expression cassette.” An “expression cassette” is polynucleotide that provides for the expression of a particular transcription unit. A transcription unit may include promoter elements and various other elements that function in the transcription of a gene or transcription unit, such as a polynucleotide encoding all or part of a therapeutic protein. An expression cassette may also be part of a larger replicating polynucleotide or expression vector.

“Isolated substantially away from other coding sequences” means that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

Expression Constructs

Expression constructs of the invention may include nucleic acids encoding a protein or polynucleotide for use in cancer therapy. In certain embodiments, genetic material may be manipulated to produce expression cassettes and expression constructs that encode the nucleic acids or inhibitors of the nucleic acids of the invention. Throughout this application, the term “expression construct” is meant to include any type of genetic construct containing a nucleic acid coding for gene products in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of therapeutic genes.

A therapeutic vector of the invention comprises a therapeutic gene for the prophylatic or therapeutic treatment of neoplastic, hyperplastic, or cancerous condition. In order to mediate the expression of a therapeutic gene in a cell, it will be necessary to transfer the therapeutic expression constructs into a cell. Such transfer may employ viral or non-viral methods of gene transfer. Gene transfer may be accomplished using a variety of techniques known in the art, including but not limited to adenovirus, various retroviruses, adeno-associated virus, vaccinia virus, canary pox virus, herpes viruses or other non-viral methods of nucleic acid delivery.

Various methods and compositions for nucleic acid transfer, both ex vivo and in vivo may be found in the following references: Carter and Flotte, 1996; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996; Xiao et al., 1996; McCown et al., 1996; Ping et al., 1996; Ridgeway, 1988; Baichwal and Sugden, 1986; Coupar et al., 1988. Other methods of gene transfer include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), naked DNA expression construct (Klein et al., 1987; Yang et al., 1990), Liposomes (Ghosh and Bachhawat, 1991; Radler et al., 1997; Nicolau et al. 1987; Kaneda et al., 1989; Kato et al., 1991) and receptor-mediated transfection (Wu and Wu, 1987; Wu and Wu, 1988).

Control Regions

Expression cassettes or constructs of the invention, encoding a therapeutic gene will typically include various control regions. These control regions typically modulate the expression of the gene of interest. Control regions include promoters, enhancers, polyadenylation signals, and translation terminators. A “promoter” refers to a DNA sequence recognized by the machinery of the cell, or introduced machinery, required to initiate the specific transcription of a gene. In particular aspects, transcription may be constitutive, inducible, and/or repressible. The phrase “under transcriptional control” means that the promoter is in the correct location and orientation in relation to the nucleic acid to control RNA polymerase initiation and expression of the gene.

In various embodiments, the human cytomegalovirus immediate early gene promoter (CMVIE), the SV40 early promoter, the Rous sarcoma virus long terminal repeat, β-actin, rat insulin promoter and glyceraldehyde-3-phosphate dehydrogenase can be used to obtain high level expression of the coding sequence of interest. The use of other viral, retroviral or mammalian cellular or bacterial phage promoters, which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Selection of a promoter that is regulated in response to specific physiologic or synthetic signals can permit inducible expression of the gene product. For example in the case where expression of a transgene, or transgenes when a multicistronic vector is utilized, is toxic to the cells in which the vector is produced in, it may be desirable to prohibit or reduce expression of one or more of the transgenes. Examples of transgenes that may be toxic to the producer cell line are pro-apoptotic and cytokine genes. Several inducible promoter systems are available for

production of viral vectors where the transgene product may be toxic. For example, the

ecdysone system (Invitrogen, Carlsbad, Calif.) and Tet-Off™ or Tet-On™ system (Clontech, Palo Alto, Calif.) are two such systems.

In some circumstances, it may be desirable to regulate expression of a transgene in a therapeutic expression vector. For example, different viral promoters with varying strengths of activity may be utilized depending on the level of expression desired. In mammalian cells, the CMV immediate early promoter if often used to provide strong transcriptional activation. Modified versions of the CMV promoter that are less potent have also been used when reduced levels of expression of the transgene are desired. When expression of a transgene in hematopoietic cells is desired, retroviral promoters such as the LTRs from MLV or MMTV are often used. Other viral promoters that may be used depending on the desired effect include SV40, RSV LTR, HIV-1 and HIV-2 LTR, adenovirus promoters such as from the E1A, E2A, or MLP region, AAV LTR, cauliflower mosaic virus, HSV-TK, and avian sarcoma virus.

Similarly tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, the following promoters may be used to target gene expression in other tissues.

Tumor specific promoters such as osteocalcin, hypoxia-responsive element (HRE), MAGE-4, CEA, alpha-fetoprotein, GRP78/BiP and tyrosinase may also be used to regulate gene expression in tumor cells.

It is envisioned that any of the above promoters alone or in combination with another may be useful according to the present invention depending on the action desired. In addition, this list of promoters should not be construed to be exhaustive or limiting, those of skill in the art will know of other promoters that may be used in conjunction with the promoters and methods disclosed herein.

Enhancers may also be utilized in construction of an expression vector. Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Polyadenylation signals may be used in therapeutic expression vectors. Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human or bovine growth hormone and SV40 polyadenylation signals. Also contemplated as an element of the expression cassette is a terminator. These elements can serve to enhance message levels and to minimize read through from the cassette into other sequences.

Multigene Constructs and IRES

In certain embodiments of the invention, the use of internal ribosome binding sites (IRES) elements are used to create multigene, polycistronic messages. IRES elements are able to bypass the ribosome scanning model of 5′-methylated, Cap-dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple genes can be efficiently expressed using a single promoter/enhancer to transcribe a single message. Any heterologous open reading frame can be linked to IRES elements. This includes genes for therapeutic proteins and selectable markers. In this way, expression of several proteins can be simultaneously engineered into a cell with a single construct and a single selectable marker.

Preparation of Nucleic Acids

In addition to the preparation of nucleic acids from a tumor sample and isolated nucleic acid may be prepared as follows. An isolated nucleic acid may be made by any technique known to one of ordinary skill in the art, such as for example, chemical synthesis, enzymatic production,

or biological production. Non-limiting examples of a synthetic nucleic acid (e.g., a synthetic oligonucleotide), include a nucleic acid made by in vitro chemical synthesis using phosphotriester, phosphite, or phosphoramidite chemistry; and solid phase techniques such as described in EP 266 032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotides may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which are incorporated herein by reference.

A non-limiting example of an enzymatically produced nucleic acid include one produced by enzymes in amplification reactions such as PCRTM (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A nonlimiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

Purification of Nucleic Acids

A nucleic acid may be purified on polyacrylamide gels, cesium chloride centrifugation gradients, affinity columns, or by any other means known to one of ordinary skill in the art (see for example, Sambrook et al., 2001, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

Nucleic Acid Segments

In certain embodiments, the nucleic acid is a nucleic acid segment. As used herein, the term “nucleic acid segment,” are smaller fragments of a nucleic acid, such as those that encode only part of the sequence of the gene in Table 1. Thus, a “nucleic acid segment” may comprise any part of a gene sequence, from about 8 nucleotides to the full length of the genes in Table 1. Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence.

Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. This algorithm would be applied to each of genes in Table 1. As used herein, a “probe” generally refers to a nucleic acid used in a detection method or composition. As used herein, a “primer” generally refers to a nucleic acid used in an extension or amplification method or composition.

Other objects, features and advantages of the present invention will become apparent from the examples. It should be understood, however, that the description and the specific examples, while indicating specific embodiments of the invention, are given by way of illustration only, since various changes and modifications within the spirit and scope of the invention will become apparent to those skilled in the art from this detailed description.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### General Methods: Patient Population

Chemosensitivity

Patients and methods used for assessment of chemosensitivity towards T/FAC are as described in detail before by Hess at al. (2006) and Ayers et al. (2004). The inventors in those studies identified a set of 193 genes that are differentially expressed between breast cancers that are highly chemotherapy sensitive and those which are less sensitive. These genes were identified by comprehensive gene expression profiling using Affymetrix U133A and B gene chips on fine needle aspiration specimens of at least 85 human breast cancers obtained at the time of diagnosis, before therapy. All patients received sequential weekly paclitaxel (P)×12 followed by 4 additional courses of 5-FU, doxorubicine, and cyclophosphamide (FAC) preoperative chemotherapy. These 193 genes, including subsets of these genes, combined with a prediction algorithm can be used to identify patients at the time of diagnosis who have better than average probability to experience complete eradication of the cancer (pathologic complete response, pCR) to T/FAC chemotherapy.

All patients were enrolled in a clinical trial at M.D. Anderson Cancer Center (protocol LAB99-402). Patients were grouped into two groups based on pathologic response outcome determined by pathologic examination of the surgically resected breast tissues after completion of six months of chemotherapy. Twenty-one of 82 patients had pathologic complete response (pCR) and 61 of 82 patients had residual disease (RD). The chemotherapy consisted of weekly paclitaxel 80 mg/m2×12 courses followed by four additional treatments with a combination of 5-fluorouracil (500 mg/m2), doxorubicin (50 mg/m2) 72-hour infusion, and cyclophosphamide (500 mg/m2) given once every 3 weeks. All patients received 24 weeks of sequential T/FAC chemotherapy and subsequently underwent lumpectomy or modified radical mastectomy with axillary node sampling as determined appropriate by the surgeon. Metallic markers had been placed under radiological guidance in the shrinking tumor bed for any patient whose tumor became <1 cm by imaging during the course of treatment. Clinical characteristics and treatment history are presented in Table 2. At the completion of neoadjuvant chemotherapy all patients had surgical resection of the tumor bed, with negative margins. Grossly visible residual cancer was measured and representative sections were submitted for histopathologic study. When there was not grossly visible residual cancer, the slices of the specimen were radiographed and all areas of radiologically and/or architecturally abnormal tissue were entirely submitted for histopathologic study. This study was approved by the institutional review board (IRB) of MDACC and all patients signed an informed consent for voluntary participation. Table 2 provides details of patient characteristics.

TABLE 2
Clinical information and demographics of the patients included
in the study (n = 82) Endocrine sensitivity

| | |
|---|---|
| Female | 82 (100%) |
| Median age | 52 years (range 29-79) |
| Race | |
| Caucasian | 56 (68%) |
| African American | 11 (13%) |
| Asian | 7 (9%) |
| Hispanic | 6 (7%) |
| Mixed | 2 (2%) |
| Histology | |
| Invasive ductal | 73 (89%) |
| Mixed ductal/lobular | 6 (7%) |
| Invasive lobular | 1 (1%) |
| Invasive mucinous | 2 (2%) |
| TNM stage | |
| T1 | 7 (9%) |
| T2 | 46 (56%) |
| T3 | 15 (18%) |
| T4 | 14 (17%) |
| N0 | 28 (34%) |
| N1 | 38 (46%) |
| N2 | 8 (10%) |
| N3 | 8 (10%) |
| Nuclear grade (BMN) | |
| 1 | 2 (2%) |
| 2 | 23 (37%) |
| 3 | 35 (61%) |
| ER positive 1 | 35 (43%) |
| ER negative | 47 (57%) |
| HER-2 positive 2 | 57 (70%) |
| HER-2 negative | 25 (30%) |
| Neoadjuvant therapy 3 | |
| Weekly T (80 mg/m2) x | 69 (84%) |
| 3-weekly T (225 mg/m | 13 (16%) |
| Pathologic complete response | 21 (26%) |
| Residual Disease (RD) | 61 (74%) |

Studies of endocrine sensitivity were conducted using different cohorts of samples: 132 patients (82 were ER-positive) from UT M.D. Anderson Cancer Center (MDACC) prior to pre-operative adjuvant chemotherapy, 18 patients from MDACC with metastatic (AJCC Stage 1V) ER-positive breast cancer, 277 patients from three different institutions (109 from Oxford, UK; 87 from Guy's Hospital, London UK; 81 from Uppsala, Sweden) who were uniformly treated with adjuvant tamoxifen, and 286 patients (209 were ER-positive) with node-negative disease from a single institution who did not receive any systemic chemotherapy treatment. At MDACC, pretreatment fine needle aspiration (FNA) samples of primary breast cancer were obtained using a 23-gauge needle and the cells from 1-2 passes were collected into a vial containing 1 ml of RNAlater™ solution (Ambion, Austin Tex.) and stored at −80° C. until use, whereas archival frozen samples were evaluated from resected, metastatic, ER-positive breast cancer. All patients signed an informed consent for voluntary participation to collect samples for research. At other institutions, fresh tissue samples of surgically resected primary breast cancer were frozen in OCT compound and stored at −80° C.

Patients in this study had invasive breast carcinoma and were characterized for estrogen receptor (ER) expression using immunohistochemistry (IHC) and/or enzyme immunoassay (ETA). Immunohistochemical (IHC) assay for ER was performed on formalin-fixed paraffin embedded (FFPE) tissue sections or Camoy' s-fixed FNA smears using the following methods: FFPE slides were first deparaffinized, then slides (FFPE or FNA) were passed through decreasing alcohol concentrations, rehydrated, treated with hydrogen peroxide (5 minutes), exposed to antigen retrieval by steaming the slides in tris-EDTA buffer at 95° C. for 45 minutes, cooled to room temperature (RT) for 20 minutes, and incubated with primary mouse monoclonal antibody 6F1 1 (Novacastra/Vector Laboratories, Burlingame, Calif.) at a dilution of 1:50 for 30 minutes at RT (Gong et al., 2004). The Envision method was employed on a Dako Autostainer instrument for the rest of the procedure according to the manufacturer's instructions (Dako Corporation, Carpenteria, Calif.). The slides were then counterstained with hematoxylin, cleared, and mounted. Appropriate negative and positive controls were included. The 96 breast cancers from OXF were ER-positive by enzyme immunoassay as previously described, containing>10 femtomoles of ER/mg protein (Blankenstein et al, 1987).

Estrogen receptor (ER) expression was characterized using immunohistochemistry (IHC) and/or enzyme immunoassay (EIA). IHC staining of ER was interpreted at MDACC as positive (P) if >10% of the tumor cells demonstrated nuclear staining, low expression (L) if <10% of the tumor cell nuclei stained, and negative (N) if there was no nuclear staining. Low expression (<10%) is reported in routine patient care as negative, but some of those patients potentially benefit from hormonal therapy (Harvey, et al., 1999).

Assessment of Bifunctional Predictors

To assess the pure prognostic value of gene sequences, we used publicly available gene expression data previously described by Wang et al (2005). This prognostic data set is available at the Gene Expression Omnibus (GEO) database (http://www.ncbi.nlm.nih.gov/geo/, accession number GSE2034) and contains gene expression information on 286 lymph node-negative patients who received no systemic adjuvant therapy. The data set includes patients with both ER-negative and ER-positive tumors but, in our analysis, we focused on the 209 patients with ER-positive tumors.

To examine the value of Tau as predictor of survival after 5 years of adjuvant tamoxifen therapy, we used data provided by the Institut Jules Bordet. This included gene expression and survival data on 267 patients with ER-positive tumors treated with adjuvant tamoxifen. Results from this dataset were previously reported (Sotiriou 2006; Loi, 2006). Data is available at the GEO database (accession number pending). The third data set consisted of 82 patients with ER positive tumors treated with preoperative chemotherapy including 12 doses of weekly paclitaxel (80 mg/m2) followed by 4 courses of 5-fluorouracil (500 mg/m2), doxorubicin (50 mg/m2) and cyclophosphamide (500 mg/m2) (T/FAC). We used this data to examine the value of Tau as a single gene marker of pathologic response to T/FAC chemotherapy. Response was dichotomized as pathologic complete response (pCR, defined as no residual invasive cancer in the breast or lymph nodes) or residual disease (RD). This data set was also previously described by Hess et al (2006). In all data sets, ER-status was determined from routine pathological assessment including immunohistochemistry or ER-ligand-binding assay for the older specimens. The cut-off for ER-positivity was 10 fmol/mg protein or >10% positive cells by immunohistochemistry. Clinical characteristics are reported in TABLE 3.

TABLE 3
Patient characteristics for endocrine sensitivity
and bifunctional prediction.

| | | Untreated | T/FAC |
|---|---|---|---|
| | Tamoxifen treated | group | neoadjuvant |
| Characteristics | group (n = 267) | (n = 209) | group (n = 82) |
| Age (years) | | | |
| Median | 63 | 53 | 51 |
| Range | 44-88 | 26-83 | 28-69 |
| T stage | | | |
| T0 | 0 | 0 | 1 (1%) |
| T1 | 108 (40%) | 111 (53%) | 4 (5%) |
| T2 | 144 (54%) | 92 (44%) | 46 (56%) |
| T3/4 | 8 (3%) | 6 (3%) | 31 (38%) |
| Unknown | 7 (3%) | 0 | 0 |
| Histologic grade | | | |
| Grade 1 | 50 (19%) | 4 (2%) | 2 (2%) |
| Grade 2 | 129 (48%) | 36 (17%) | 47 (57%) |
| Grade 3 | 43 (16%) | 102 (49%) | 33 (40%) |
| Unknown | 45 (17%) | 67 (32%) | 0 |
| Lymph node status | | | |
| Positive | 140 (52%) | 0 | 55 (67%) |
| Negative | 115 (43%) | 209 (100%) | 27 (33%) |
| unknown | 12 94%) | 0 | 0 |
| AJCC stage | | | |
| Stage I | 55 (21%) | 111 (53%) | 1 (1%) |
| Stage II | 189 (71%) | 96 (46%) | 42 (51%) |
| Stage III | 7 (3%) | 2 (1%) | 39 (48%) |
| Unknown | 16 (6%) | 0 | 0 |
| ER status | | | |
| Positive | 267 (100%) | 209 (100%) | 48 (59%) |
| Negative | 0 | 0 | 31 (38%) |
| Unknown | 0 | 0 | 3 (3%) |
| PR status | | | |
| Positive | 128 (48%) | 0 | 48 (59%) |
| Negative | 26 (10%) | 0 | 31 (38%) |
| Unknown | 113 (42%) | 209 (100%) | 3 (3%) |
| Metastases within 5 | | | |
| years | | | |
| Yes | 38 (14%) | 68 (33%) | 8 (10%) |
| No | 160 (60%) | 135 (64%) | 69 (84%) |
| Censored | 69 (26%) | 6 (3%) | 5 (6%) |

RNA Extraction and Gene Expression Profiling.

RNA was extracted from the FNA samples using the RNAeasy Kit™ (Qiagen, Valencia Calif.). The amount and quality of RNA was assessed with DU-640 U.V. Spectrophotometer (Beckman Coulter, Fullerton, Calif.) and it was considered adequate for further analysis if the OD260/280 ratio was ≧1.8 and the total RNA yield was ≧1.0 μg. RNA was extracted from the tissue samples using Trizol (InVitrogen, Carlsbad, Calif.) according to the manufacturer's instructions. The quality of the RNA was assessed based on the RNA profile generated by the Bioanalyzer (Agilent Technologies, Palo Alto, Calif.). Differences in the cellular composition of the FNA and tissue samples have been reported previously (Symmans, et al. 2003). In brief, FNA samples on average contain 80% neoplastic cells, 15% leukocytes, and very few (<5%) non-lymphoid stromal cells (endothelial cells, fibroblasts, myofibroblasts, and adipocytes), whereas tissue samples on average contain 50% neoplastic cells, 30% non-lymphoid stromal cells, and 20% leukocytes (Symmans, et al. 2003). A standard T7 amplification protocol was used to generate cRNA for hybridization to the microarray. No second round amplification was performed. Briefly, mRNA sequences in the total RNA from each sample were reverse-transcribed with SuperScript II in the presence of T7-(dT)24 primer to produce cDNA. Second-strand cDNA synthesis was performed in the presence of DNA Polymerase I, DNA ligase, and Rnase H. The double-stranded cDNA was blunt-ended using T4 DNA polymerase and purified by phenol/chloroform extraction. Transcription of double-stranded cDNA into cRNA was performed in the presence of biotin-ribonucleotides using the BioArray High Yield RNA transcript labeling kit (Enzo Laboratories). Biotin-labeled cRNA was purified using Qiagen RNAeasy columns (Qiagen Inc.), quantified and fragmented at 94° C. for 35 minutes in the presence of 1× fragmentation buffer. Fragmented cRNA from each sample was hybridized to each Affymetrix U133A gene chip, overnight at 42° C. The U133A chip contains 22,215 different probe sets that correspond to 13,739 human UniGene clusters (genes). Hybridization cocktail was prepared as described in the Affymetrix technical manual. dCHIP Vi 0.3 (http://dchip.org) software was used to generate probe level intensities and quality measures including median intensity, % of probe set outliers and % of single probe outliers for each chip.

Gene Expression Analysis.

All three gene expression datasets were obtained using Affimetrix U133A Gene Chip following standard operating procedures. Additional details on sample processing, RNA extraction and hybridization are available through the original publications (Loi, 2006; Hess, 2006; Wang 2005). Surgical biopsy specimens were used for microarray profiling in the prognostic and the adjuvant endocrine data sets, whereas fine needle aspiration material was used to generate the MDACC neoadjuvant chemotherapy data. Only the normalized probe set intensities were available for downloading and analysis from the prognostic data set. For the adjuvant endocrine and for the MDACC datasets, the raw intensity files (CEL) from each microarray were available. We normalized the gene expression data using the Affymetrix dChip V1.3 software to a single reference array. The normalization files are available online at http://bioinformatics.mdanderson.org/pubdata.html (last visited on Oct. 21, 2006). After normalization, the 75th percentile of pixel level was used as the intensity level for each feature on a microarray. Multiple features representing each probe set were aggregated using the perfect match (PM) model to form a single measure of intensity for the probe set.

Tau Gene Expression

A single Affymetrix probe set, 203929_s_at, was used as a measure of Tau expression. This particular probe set was selected from the 4 distinct Tau probe sets that are present on the U133A chip because it targets the most 3′ end of the transcript and it showed the highest mean expression in 2 of the 3 data sets. It also discriminated effectively between patients with pathologic complete response (pCR) and those with residual invasive cancer (RD) after neoadjuvant chemotherapy in our previously published work (Rouzier, 2005).

Endocrine Sensitivity and ER Reporter Genes

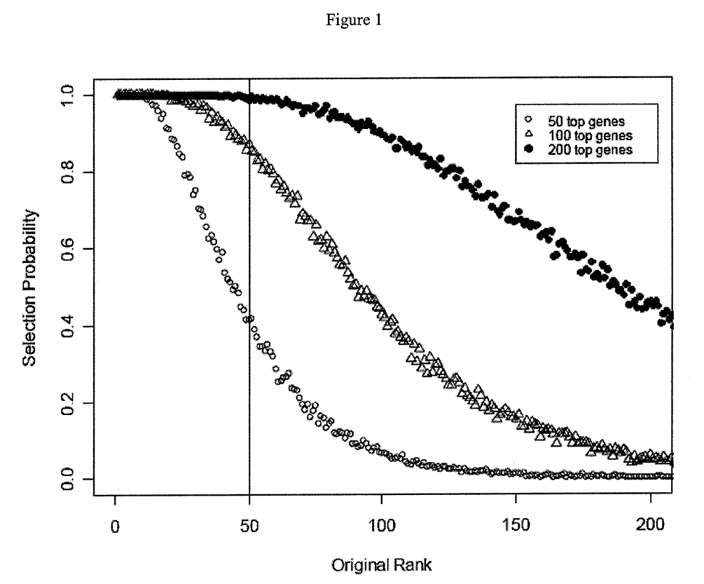

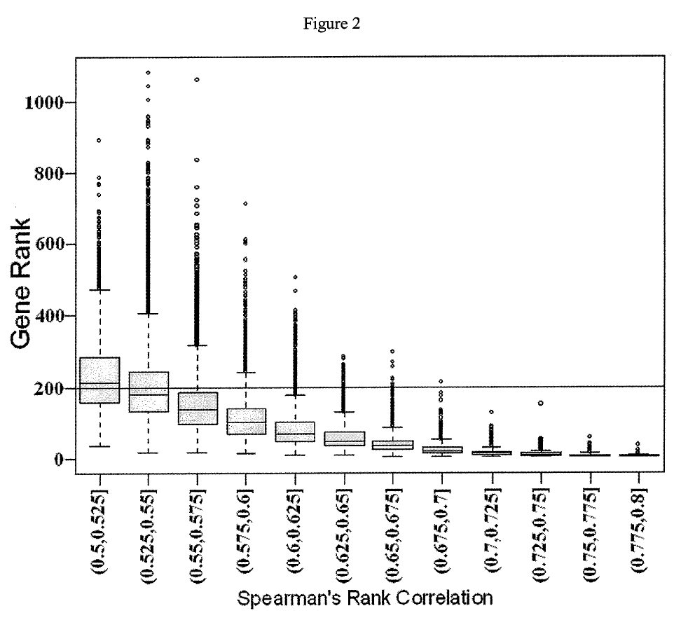

ER reporter genes were defined from an independent public dataset of Affymetrix U133A transcriptional profiles from 286 node-negative breast cancer samples (Wang et al., 2005). Expression data had been normalized to an average probe set intensity of 600 per array (Wang et al., 2005). The dataset was filtered to include 9789 probe sets with most variable expression, where P₀≧5, P₇₅-P₂₅≧100, and P₉₅/P₅≧3 (P_{q} is the qth percentile of intensity for each probe set). Those were ranked by Spearman's rho (Kendall and Gibbons, 1990) with ER mRNA (ESR1 probe set 205225_at) expression, of which 2217 probe sets were significantly and positively associated with ESR1 (t-test of correlation coefficients with one-sided significance level of 99.9% and estimated false discovery rate (FDR) of 0.45%). The size of the reporter gene set was then determined by a bootstrap-based method that accounts for sampling variability in the correlation coefficient and in the resulting probe sets rankings (Pepe et al., 2003). The entire dataset was re-sampled 1000 times with replacement at the subject level (i.e. when one of the 286 subjects was selected in the bootstrap sample, the 2217 candidate probe sets from that subject were included in the dataset). Each probe set was ranked according to its correlation with ESR1 in each bootstrap dataset. The probability (P) of selection for each probe set (g) in a reporter gene set of defined length (k) was calculated as P[Rank(g)≦k]. A similar computation provided estimates of the power to detect the truly co-expressed genes from a study of a given size (Pepe et al., 2003). Genes that are truly co-expressed with ESR1 have selection probabilities close to 1, but the selection probability diminishes quickly for lower order probe sets (FIG. 1). The probability of selecting the top 50 ER-associated probes would be 98.5% if the ER reporter gene list included 200 probes, 87.0% if 100 probes, and 41.3% if 50 probes (FIG. 1). An ER reporter list with 200 top-ranking probes would include the top 50 probes with 98.5% probability and the top 100 probes with about 93% probability (FIG. 1). The distribution of ranks is very tight for genes that are strongly correlated with ESR1 having median ranks close to 1 (FIG. 2). However, both the median rank and the variance of the distribution of ranks increase for genes that are moderately correlated with ESR1. The gene ranks for genes with Spearman's rho>0.65 are less than 200 with the exception of a few outliers (FIG. 2, red line). Therefore as opposed to selecting the reporter genes by choosing an arbitrary cutoff on the correlation coefficient, this approach identifies the 100 genes that are most-strongly correlated with ESR1 with high power (>93%). The size of the reporter gene set was selected to be 200 probe sets, based on the bootstrap-estimated selection probabilities (FIG. 1) and the requirement to detect the top 100 truly co-expressed genes with >90% power. The original dataset was re-sampled with replacement at the subject level (i.e. when one of the 286 subjects was selected in the bootstrap sample, the 2217 candidate probe sets from that subject were included in the dataset to generate 1000 different bootstrap datasets. Each candidate probe set was ranked according to its correlation with ESR1 within each bootstrap dataset and the degree of confidence in the ranking of each probe set was quantified in terms of the selection probability, Pg(k) The probability (P) of selection for each probe set (g) in a reporter gene set of defined length (k) was calculated as P[Rank(g)]≦k. The 200 ER-activity-related genes are listed in Table 4 below.

TABLE 4
Reporter genes for ER-related genomic activity

| Rank | Probe Set ID | Unigene ID | Gene Symbol | Rs | Pg(200) |
|---|---|---|---|---|---|
| 1 | 209603_at | 169946 | GATA3 | 0.783 | 1.000 |
| 2 | 215304_at | 159264 | | 0.779 | 1.000 |
| 3 | 218195_at | 15929 | C6orf211 | 0.774 | 1.000 |
| 4 | 212956_at | 411317 | KIAA0882 | 0.771 | 1.000 |
| 5 | 209604_s_at | 169946 | GATA3 | 0.764 | 1.000 |
| 6 | 202088_at | 79136 | SLC39A6 | 0.757 | 1.000 |
| 7 | 209602_s_at | 169946 | GATA3 | 0.749 | 1.000 |
| 8 | 212496_s_at | 301011 | JMJD2B | 0.733 | 1.000 |
| 9 | 212960_at | 411317 | KIAA0882 | 0.724 | 1.000 |
| 10 | 215867_x_at | 5344 | AP1G1 | 0.724 | 1.000 |
| 11 | 214164_x_at | 512620 | CA12 | 0.721 | 1.000 |
| 12 | 203963_at | 512620 | CA12 | 0.719 | 1.000 |
| 13 | 41660_at | 252387 | CELSR1 | 0.709 | 1.000 |
| 14 | 218259_at | 151076 | MRTF-B | 0.695 | 1.000 |
| 15 | 204667_at | 163484 | FOXA1 | 0.689 | 1.000 |
| 16 | 211712_s_at | 430324 | ANXA9 | 0.684 | 1.000 |
| 17 | 218532_s_at | 82273 | FLJ20152 | 0.677 | 1.000 |
| 18 | 212970_at | 15740 | FLJ14001 | 0.677 | 1.000 |
| 19 | 209459_s_at | 1588 | ABAT | 0.676 | 0.999 |
| 20 | 204508_s_at | 512620 | CA12 | 0.675 | 1.000 |
| 21 | 218976_at | 260720 | DNAJC12 | 0.673 | 0.998 |
| 22 | 217838_s_at | 241471 | EVL | 0.673 | 1.000 |
| 23 | 218211_s_at | 297405 | MLPH | 0.669 | 1.000 |
| 24 | 222275_at | 124165 | MRPS30 | 0.666 | 1.000 |
| 25 | 218471_s_at | 129213 | BBS1 | 0.666 | 0.999 |
| 26 | 214053_at | 7888 | | 0.666 | 0.999 |
| 27 | 203438_at | 155223 | STC2 | 0.664 | 1.000 |
| 28 | 213234_at | 6189 | KIAA1467 | 0.664 | 0.999 |
| 29 | 219197_s_at | 435861 | SCUBE2 | 0.657 | 0.999 |
| 30 | 212692_s_at | 209846 | LRBA | 0.657 | 0.999 |
| 31 | 200711_s_at | 171626 | SKP1A | 0.654 | 1.000 |
| 32 | 205074_at | 15813 | SLC22A5 | 0.653 | 1.000 |
| 33 | 203685_at | 501181 | BCL2 | 0.653 | 1.000 |
| 34 | 209460_at | 1588 | ABAT | 0.653 | 0.999 |
| 35 | 222125_s_at | 271224 | PH-4 | 0.651 | 1.000 |
| 36 | 204798_at | 407830 | MYB | 0.651 | 0.999 |
| 37 | 212985_at | 15740 | FLJ14001 | 0.648 | 1.000 |
| 38 | 203929_s_at | 101174 | MAPT | 0.647 | 0.998 |
| 39 | 202089_s_at | 79136 | SLC39A6 | 0.642 | 0.997 |
| 40 | 205696_s_at | 444372 | GFRA1 | 0.639 | 0.997 |
| 41 | 209681_at | 30246 | SLC19A2 | 0.637 | 0.999 |
| 42 | 212495_at | 301011 | JMJD2B | 0.637 | 0.999 |
| 43 | 218510_x_at | 82273 | FLJ20152 | 0.634 | 0.995 |
| 44 | 208682_s_at | 376719 | MAGED2 | 0.632 | 0.994 |
| 45 | 212195_at | 529772 | | 0.630 | 0.997 |
| 46 | 51192)at | 29173 | SSH-3 | 0.630 | 0.999 |
| 47 | 40016_g_at | 212787 | KIAA0303 | 0.628 | 0.997 |
| 48 | 212638_s_at | 450060 | WWP1 | 0.627 | 0.994 |
| 49 | 218692_at | 354793 | FLJ20366 | 0.624 | 0.991 |
| 50 | 213077_at | 283283 | FLJ21940 | 0.623 | 0.985 |
| 51 | 203439_s_at | 155223 | STC2 | 0.623 | 0.995 |
| 52 | 212441_at | 79276 | KIAA0232 | 0.622 | 0.988 |
| 53 | 210652_s_at | 112949 | C1orf34 | 0.621 | 0.990 |
| 54 | 219981_x_at | 288995 | ZNF587 | 0.620 | 0.984 |
| 55 | 205186_at | 406050 | DNALI1 | 0.620 | 0.990 |
| 56 | 213627_at | 376719 | MAGED2 | 0.620 | 0.987 |
| 57 | 200670_at | 437638 | XBP1 | 0.617 | 0.985 |
| 58 | 218437_s_at | 30824 | LZTFL1 | 0.617 | 0.987 |
| 59 | 206754_s_at | 1360 | CYP2B6 | 0.616 | 0.985 |
| 60 | 209696_at | 360509 | FBP1 | 0.616 | 0.987 |
| 61 | 201826_s_at | 238126 | CGI-49 | 0.615 | 0.984 |
| 62 | 219833_s_at | 446047 | EFHC1 | 0.610 | 0.975 |
| 63 | 203928_x_at | 101174 | MAPT | 0.610 | 0.976 |
| 64 | 216092_s_at | 22891 | SLC7A8 | 0.609 | 0.985 |
| 65 | 200810_s_at | 437351 | CIRBP | 0.609 | 0.977 |
| 66 | 204811_s_at | 389415 | CACNA2D2 | 0.609 | 0.968 |
| 67 | 44654_at | 294005 | G6PC3 | 0.609 | 0.974 |
| 68 | 202371_at | 194329 | FLJ21174 | 0.608 | 0.970 |
| 69 | 209173_at | 226391 | AGR2 | 0.607 | 0.971 |
| 70 | 212196_at | 529772 | | 0.606 | 0.953 |
| 71 | 210720_s_at | 324104 | APBA2BP | 0.606 | 0.965 |
| 72 | 204497_at | 20196 | ADCY9 | 0.605 | 0.965 |
| 73 | 214440_at | 155956 | NAT1 | 0.604 | 0.960 |
| 74 | 205099_at | 350470 | TFF1 | 0.603 | 0.964 |
| 75 | 204862_s_at | 81687 | NME3 | 0.601 | 0.971 |
| 76 | 219562_at | 3797 | RAB26 | 0.600 | 0.949 |
| 77 | 50965_at | 3797 | RAB26 | 0.599 | 0.951 |
| 78 | 218966_at | 111782 | MYO5C | 0.598 | 0.961 |
| 79 | 217979_at | 364544 | TM4SF13 | 0.596 | 0.972 |
| 80 | 209759_s_at | 403436 | DCI | 0.596 | 0.938 |
| 81 | 212637_s_at | 450060 | WWP1 | 0.594 | 0.951 |
| 82 | 218094_s_at | 256086 | C20orf35 | 0.592 | 0.954 |
| 83 | 219222_at | 11916 | RBKS | 0.592 | 0.941 |
| 84 | 202121_s_at | 12107 | BC-2 | 0.591 | 0.940 |
| 85 | 215001_s_at | 442669 | GLUL | 0.591 | 0.940 |
| 86 | 210085_s_at | 430324 | ANXA9 | 0.590 | 0.934 |
| 87 | 210958_s_at | 212787 | KIAA0303 | 0.589 | 0.940 |
| 88 | 201596_x_at | 406013 | KRT18 | 0.588 | 0.928 |
| 89 | 212209_at | 435249 | THRAP2 | 0.587 | 0.923 |
| 90 | 221139_s_at | 279815 | CSAD | 0.586 | 0.924 |
| 91 | 201384_s_at | 458271 | M17S2 | 0.586 | 0.910 |
| 92 | 213283_s_at | 416358 | SALL2 | 0.586 | 0.927 |
| 93 | 202908_at | 26077 | WFS1 | 0.585 | 0.917 |
| 94 | 219786_at | 121378 | MTL5 | 0.585 | 0.918 |
| 95 | 214109_at | 209846 | LRBA | 0.584 | 0.930 |
| 96 | 203791_at | 181042 | DMXL1 | 0.583 | 0.914 |
| 97 | 205012-s_at | 155482 | HAGH | 0.583 | 0.903 |
| 98 | 212492_s_at | 301011 | JMJD2B | 0.582 | 0.902 |
| 99 | 218026_at | 16059 | HSPC009 | 0.579 | 0.905 |
| 100 | 210272_at | 1360 | CYP2B6 | 0.579 | 0.897 |
| 101 | 204199_at | 432842 | RALGPS1 | 0.577 | 0.892 |
| 102 | 202752_x_at | 22891 | SLC7A8 | 0.577 | 0.886 |
| 103 | 217645_at | 531103 | | 0.576 | 0.882 |
| 104 | 213419_at | 324125 | APBB2 | 0.576 | 0.888 |
| 105 | 219919_s_at | 29173 | SSH-3 | 0.575 | 0.861 |
| 106 | 213365_at | 248437 | MGC16943 | 0.574 | 0.861 |
| 107 | 219206_x_at | 126372 | CGI-119 | 0.574 | 0.883 |
| 108 | 221751_at | 388400 | PANK3 | 0.573 | 0.875 |
| 109 | 211596_s_at | 528353 | LRIG1 | 0.572 | 0.863 |
| 110 | 221963_x_at | 356530 | | 0.572 | 0.867 |
| 111 | 202641_at | 182215 | ARL3 | 0.572 | 0.850 |
| 112 | 201754_at | 351875 | COX6C | 0.571 | 0.857 |
| 113 | 219741-x_at | 515644 | ZNF552 | 0.569 | 0.848 |
| 114 | 209224_s_at | | NDUFA2 | 0.568 | 0.862 |
| 115 | 212099_at | 406064 | RHOB | 0.568 | 0.836 |
| 116 | 205794_s_at | 292511 | NOVA1 | 0.568 | 0.836 |
| 117 | 219913_s_at | 171342 | CRNKL1 | 0.568 | 0.816 |
| 118 | 204934_s_at | 432750 | HPN | 0.567 | 0.830 |
| 119 | 209341-s-at | 413513 | IKBKB | 0.567 | 0.816 |
| 120 | 204231_s_at | 528334 | FAAH | 0.567 | 0.817 |
| 121 | 203571_s_at | 511763 | C10orf116 | 0.567 | 0.807 |
| 122 | 204045_at | 95243 | TCEAL1 | 0.566 | 0.833 |
| 123 | 202636_at | 147159 | RNF103 | 0.566 | 0.788 |
| 124 | 202962_at | 15711 | KIF13B | 0.565 | 0.798 |
| 125 | 208865_at | 318381 | CSNK1A1 | 0.563 | 0.801 |
| 126 | 201825_s_at | 238126 | CGI-49 | 0.563 | 0.806 |
| 127 | 219686_at | 58241 | STK32B | 0.562 | 0.806 |
| 128 | 57540_at | 11916 | RBKS | 0.560 | 0.782 |
| 129 | 212416_at | 31218 | SCAMP1 | 0.559 | 0.801 |
| 130 | 201170_s_at | 171825 | BHLHB2 | 0.559 | 0.758 |
| 131 | 40093_at | 155048 | LU | 0.558 | 0.773 |
| 132 | 219414_at | 12079 | CLSTN2 | 0.557 | 0.761 |
| 133 | 209623_at | 167531 | MCCC2 | 0.556 | 0.758 |
| 134 | 202772_at | 444925 | HMGCL | 0.555 | 0.752 |
| 135 | 208517_x_at | 446567 | BTF3 | 0.553 | 0.734 |
| 136 | 213018_at | 21145 | ODAG | 0.552 | 0.764 |
| 137 | 204703_at | 251328 | TTC10 | 0.551 | 0.731 |
| 138 | 203801_at | 247324 | MRPS14 | 0.551 | 0.730 |
| 139 | 203246_s_at | 437083 | TUSC4 | 0.550 | 0.733 |
| 140 | 218769-s_at | 239154 | ANKRA2 | 0.549 | 0.740 |
| 141 | 203476_at | 82128 | TPBG | 0.549 | 0.706 |
| 142 | 217770_at | 437388 | PIGT | 0.548 | 0.736 |
| 143 | 35666_at | 32981 | SEMA3F | 0.547 | 0.694 |
| 144 | 212508_at | 24719 | MOAP1 | 0.546 | 0.686 |
| 145 | 208712_at | 371468 | CCND1 | 0.545 | 0.703 |
| 146 | 204863_s_at | 71968 | IL6ST | 0.544 | 0.710 |
| 147 | 204284_at | 303090 | PPP1R3C | 0.544 | 0.672 |
| 148 | 203628_at | 239176 | IGF1R | 0.544 | 0.674 |
| 149 | 200719_at | 171626 | SKP1A | 0.544 | 0.668 |
| 150 | 214919_s_at | | MASK-BP3 | 0.544 | 0.669 |
| 151 | 205376_at | 153687 | INPP4B | 0.544 | 0.691 |
| 152 | 202263_at | 334832 | CYB5R1 | 0.543 | 0.674 |
| 153 | 218450_at | 294133 | HEBP1 | 0.543 | 0.660 |
| 154 | 213285_at | 146180 | LOC161291 | 0.543 | 0.666 |
| 155 | 209740_s_at | 264 | DXS1283E | 0.543 | 0.653 |
| 156 | 205380_at | 15456 | PDZK1 | 0.543 | 0.661 |
| 157 | 203144_s-at | 368916 | KIAA0040 | 0.543 | 0.656 |
| 158 | 214552_s_at | 390163 | RABEP1 | 0.542 | 0.660 |
| 159 | 202814_s_at | 15299 | HIS1 | 0.540 | 0.629 |
| 160 | 205776_at | 396595 | FMO5 | 0.539 | 0.633 |
| 161 | 217906_at | 415236 | KLHDC2 | 0.539 | 0.640 |
| 162 | 212148_at | 408222 | PBX1 | 0.539 | 0.620 |
| 163 | 220581_at | 287738 | C6orf97 | 0.538 | 0.643 |
| 164 | 200811_at | 437351 | CIRBP | 0.538 | 0.574 |
| 165 | 217894_at | 239155 | KCTD3 | 0.538 | 0.580 |
| 166 | 206197_at | 72050 | NME5 | 0.537 | 0.610 |
| 167 | 202454_s_at | 306251 | ERBB3 | 0.537 | 0.614 |
| 168 | 218394_at | 22795 | FLJ22386 | 0.535 | 0.601 |
| 169 | 201413_at | 356894 | HSD17B4 | 0.535 | 0.593 |
| 170 | 40569_at | 458361 | ZNF42 | 0.535 | 0.574 |
| 171 | 221856_s_at | 3346 | FLJ11280 | 0.535 | 0.576 |
| 172 | 210336_x_at | 458361 | ZNF42 | 0.534 | 0.584 |
| 173 | 211621_at | 99915 | AR | 0.533 | 0.573 |
| 174 | 204623_at | 82961 | TFF3 | 0.533 | 0.533 |
| 175 | 40148_at | 324125 | APBB2 | 0.533 | 0.581 |
| 176 | 212446_s_at | 387400 | LASS6 | 0.532 | 0.543 |
| 177 | 210735_s_at | 279916 | CA12 | 0.531 | 0.540 |
| 178 | 214924_s_at | 457063 | OIP106 | 0.531 | 0.561 |
| 179 | 203071_at | 82222 | SEMA3B | 0.531 | 0.522 |
| 180 | 213527_s_at | 301463 | LOC146542 | 0.530 | 0.531 |
| 181 | 208617_s_at | 82911 | PTP4A2 | 0.530 | 0.517 |
| 182 | 213249_at | 76798 | FBXL7 | 0.529 | 0.552 |
| 183 | 205645-at | 334168 | REPS2 | 0.529 | 0.520 |
| 184 | 208788_at | 343667 | ELOVL5 | 0.529 | 0.543 |
| 185 | 205769_at | 11729 | SLC27A2 | 0.528 | 0.501 |
| 186 | 213712_at | 246107 | ELOVL2 | 0.528 | 0.510 |
| 187 | 212697_at | 432850 | LOC162427 | 0.528 | 0.503 |
| 188 | 219900_s_at | 435303 | FLJ20626 | 0.528 | 0.485 |
| 189 | 213832_at | 23729 | | 0.527 | 0.490 |
| 190 | 213049_at | 167031 | GARNL1 | 0.527 | 0.474 |
| 191 | 59437_at | 414028 | C9orf116 | 0.527 | 0.504 |
| 192 | 204072_s_at | 390874 | 13CDNA73 | 0.526 | 0.451 |
| 193 | 210108_at | 399966 | CACNA1D | 0.526 | 0.489 |
| 194 | 214855_s_at | 167031 | GARNL1 | 0.525 | 0.459 |
| 195 | 209662_at | 528302 | CETN3 | 0.525 | 0.441 |
| 196 | 219687_at | 58650 | MART2 | 0.525 | 0.470 |
| 197 | 217191_x_at | | COX6CP1 | 0.524 | 0.440 |
| 198 | 203538_at | 13572 | CAMLG | 0.524 | 0.442 |
| 199 | 213702_x_at | 324808 | ASAH1 | 0.522 | 0.456 |
| 200 | 212744_at | 26471 | BBS4 | 0.522 | 0.458 |

Identification of Bifunctional Predictors

To identify informative chemosensitivity genes differentially expressed between cases with pCR and those with residual disease, genes were ordered by p-values obtained with two-sample, unequal-variance t-tests (Ayers et al, 2004; Hess et al, 2006). Genes involved in estrogen activity and endocrine sensitivity were identified as described in the previous section. To identify bifunctional prediction by a gene sequence, we rank-ordered all 200 genes correlated with ESR1 (Table 4) but with negative correlation with chemosensitivity indicating a downregulation in correlation with pCR. This provided the list of 21 genes listed in Table 1 with Tau showing strongest negative correlation with pCR. Tau was characterized further as described below.

Statistical Analysis for Tau Expression.

The correlation between Tau expression and ER status and between Tau expression and pCR were assessed by two-sample Wilcoxon rank sum test. This test was used because of the non-normal distribution of Tau. The prognostic value of Tau in untreated and tamoxifen-treated patients was assessed by examining the association between Tau expression and the occurrence of distant metastases using the Wilcoxon test. To assess if covariates including grade, tumor size and nodal status were associated with Tau levels we performed multivariate linear regression analysis. Kaplan-Meier survival curves were compared using the Log-rank test. In order to generate Kaplan-Meier curves, patients had to be assigned to various Tau expression groups. However, no cut off points have been established previously to assign low or high Tau status to cases based on microarray results. Furthermore, normalized expression values differed between the data sets, particularly for the prognostic data set that used different normalization procedure. In the absence of accepted cut off values and considering the non-normal expression distribution of Tau, we grouped patients by tertiles and quartiles. The R statistical environment was used for all statistical analyses (R Development Core Team, 2006).

Results

Characterization of Tau for Gene Expression and Response Prediction

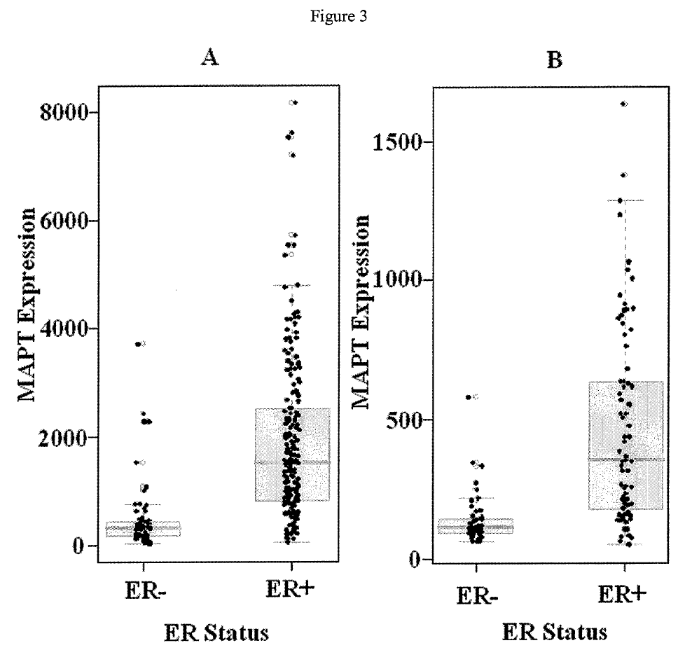

Tau expression was examined in relation to ER status in two distinct cohorts of patients. The first included public microarray data from 286 patients including 209 ER-positive and 77 ER-negative cases. Gene expression data was derived from analysis of surgical resection tissue and the ER status was assigned to each case by the original investigators based on routine pathology results (Wang, 2005). The second cohort included 133 cases from M.D. Anderson Cancer Center, including 82 ER-positive and 51 ER-negative tumors. Gene expression data was generated from fine needle aspiration specimens. FIG. 3 illustrates the results. In both data sets Tau expression was significantly lower in ER-negative cancers compared to ER-positive cancers. The median expression was 300 normalised intensity units (range: 14-3690) in the ER negative and 1511 (range: 39-8139) in ER-positive tumors (p<0.0001) in the Wang data set. It was 112 (range: 61-579) and 350 (range: 51-1628), respectively (p<0.0001) in the MDACC data set. The differences in intensity scale are due to differences in the normalization procedures applied to the two data sets.

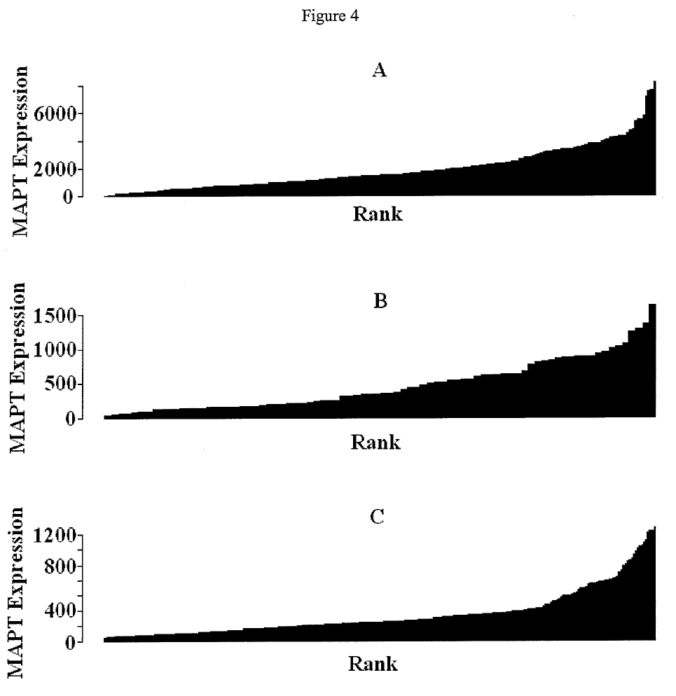

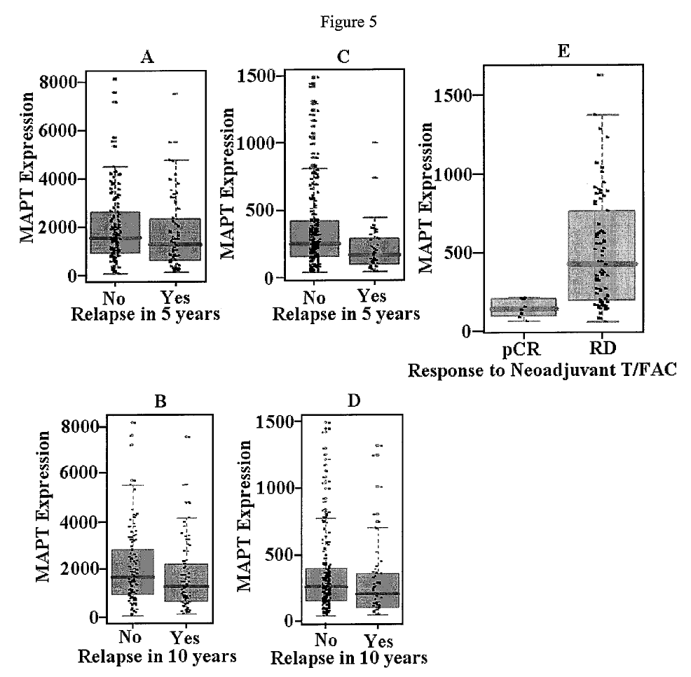

Next we examined Tau expression within ER-positive breast cancers only. FIG. 4 illustrates that there is substantial variation in Tau expression within ER-positive cancers and the expression distribution is non-normal. This indicates that within each distinct data set there is a minority of ER-positive cancers that show very high expression levels of Tau compared to the rest of cases.

### Example 2

### Prognostic Value of Tau in ER-Positive, Node-Negative Disease in the Absence of Systemic Adjuvant Therapy

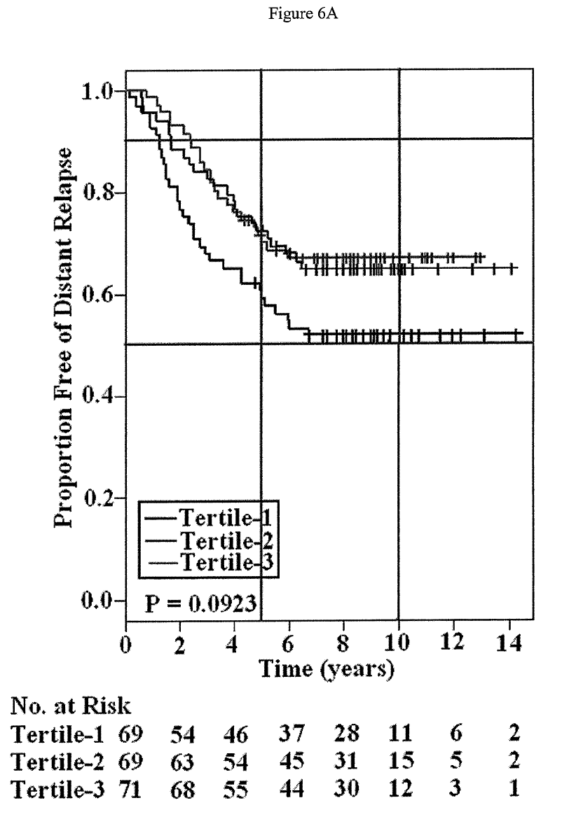

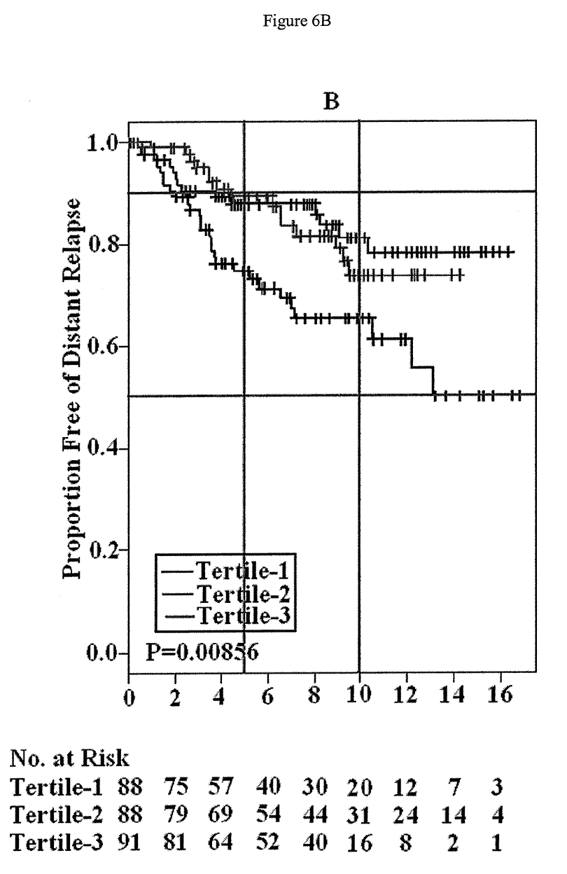

We first assessed the prognostic value of Tau mRNA expression in patients with nodenegative, ER-positive disease who received no adjuvant systemic therapy. Of the 209 patients included in this analysis, 127 had more than 5 years follow-up and 34 patients had >10 years of follow-up. There was a statistically non-significant trend for lower Tau expression in those who relapsed in the first 5 years of follow up (median 1300, SD: 1476) compared to those who did not (median 1558, SD: 1498), p=0.09 (FIG. 5A). Interestingly, this trend reached significance by 10 years of follow up, p=0.025 (FIG. 5B). We also performed Kaplan Meier survival analysis and grouped patients into tertiles and quartiles of Tau expression. There was a statistically non-significant trend towards better survival in patients in the top two tertiles (top 66%, p=0.09, FIG. 6A) and quartiles (top 50%, p=0.14, supplementary FIG. 6B) compared to patients with lower levels of Tau expression. These results indicate that high Tau mRNA expression has a borderline association with better prognosis. However, it is not a significant prognostic marker in the absence of systemic adjuvant therapy.

### Example 3

### Prognostic Value of Tau in ER-Positive Breast Cancer Treated with Adjuvant Tamoxifen

We examined Tau expression in 267 ER-positive tumors from patients who received 5 years of adjuvant tamoxifen therapy. The median follow up for these patients was 101 months (range 20-171). FIG. 5C shows that Tau expression was significantly higher (p=0.005) in patients who did not have a distant metastatic recurrence in 5-years (n=229, median=258, SD: 305) compared to those who did (n=38, median=168, SD:190). The same positive correlation between Tau levels and better recurrence-free survival remained significant at 10 years of follow up, FIG. 5D (p=0.025). Multivariate linear regression analysis including grade, tumor size and nodal status showed a significant association between Tau expression and grade. Tau expression was lower in higher grade tumors (p=0.025). Kaplan Meier survival analysis of patients grouped into tertiles and quartiles of Tau expression confirmed significantly better survival for patients in the upper 2 tertiles (p=0.008), FIG. 6D. Multivariate Cox regression analysis including age, tumor size, nodal status, ER expression and Tau indicated that Tau remains a significant independent predictor of outcome (TABLE 5).

Overall, these data indicate that patients with high Tau expression have good prognosis with adjuvant endocrine therapy. On the other hand, patients with the lowest 1/3 of Tau expression values have unacceptably high rates of relapse both at 5 and at 10 years post surgery.

TABLE 5
Multivariate analysis of Tau as predictor of disease free survival in
tamoxifen treated ER-positive breast cancer and as a predictor
or pCR in paclitaxel/FAC treated patients.

| | Tamoxifen§ | | T/FAC† | |
|---|---|---|---|---|
| Variable | HR (95% CI) | p | OR (95% CI) | P |
| Age (>mediam | 0.83 (0.24-2.94) | 0.77 | 0.96 (0.16-5.74) | 0.96 |
| vs ≦median) | | | | |
| T Stage | 2.23 (1.04-4.78) | 0.04 | 0.44 (0.03-7.21) | 0.57 |
| (2-4 cs 1) | | | | |
| Node | 1.20 (0.61-2.38) | 0.60 | 0.52 (0.08-3.43) | 0.50 |
| (pos vs negative) | | | | |
| ESR1 | 0.86 (0.58-1.29) | 0.47 | 0.68 (0.17-2.68) | 0.58 |
| (log-transformed) | | | | |
| MAPT | 0.60 (0.38-0.94) | 0.03 | 0.14 (0.03-0.59) | 0.008 |
| (log-transformed) | | | | |

§For the tamoxifen treated cohort, the results are from a multivariate Cox regression analysis of distant relapse data up to 5 yrs (relapse events occurring after 5 years were included in the analysis as censored observations)
†For the T/FAC treated cohort, the results shown are from a multivariate logistic regression model of pCR.

### Example 4

### Tau as Predictor of Response to Preoperative Paclitaxel/FAC Chemotherapy in ER-Positive Breast Cancer

In an earlier study including both ER-positive and ER-negative cases, we reported that low Tau expression was associated with higher rates of pCR to preoperative T/FAC chemotherapy (Rouzier, 2005). In the current study we examined the same hypothesis focusing on ER-positive breast cancers only. A total of 82 cases was included in the current analysis and this included 35 cases from the original study. FIG. 5E shows that the median Tau expression was 143 (SD: 58) in cases with pCR (n=7) compared to 429 (SD: 353) in cases with residual cancer (n=75) after chemotherapy (p<0.001). The pCR rate was 19% (7 of 27) in the lowest tertile of Tau expression, 3% (2 of 27) in the median tertile and 0% (0 of 28) in the highest tertile. Multivariate logistic regression analysis including age, tumor size, nodal status, ER expression and Tau indicated that Tau remains a significant independent predictor of pCR (TABLE 5). These results confirm our previous report that low Tau expression identifies a subset of breast cancers that are particularly sensitive to paclitaxel-containing neoadjuvant chemotherapy. This observation is particularly strong in ER-positive breast cancers.

We also observed a positive correlation between Tau expression and distant recurrence free survival after adjuvant tamoxifen therapy. Patients with no recurrence had higher Tau levels. This is consistent with the hypothesis that Tau may be an indicator of active estrogen signaling and is therefore a marker of sensitivity to anti-estrogen therapy. This hypothesis is also supported by laboratory evidence. Tau is an estrogen-induced gene in several in vitro cell line models (Ma, 1993; Matsuno, 1997). Tau expression showed only borderline significant association with better prognosis in the absence of any systemic adjuvant therapy in ER-positive breast cancer. This indicates that it is not a powerful prognostic marker but it is a bifunctional predictor of benefit from endocrine and chemotherapies in ER-positive breast cancer. It predicts opposite responses to these two important treatment modalities. High Tau mRNA expression in ER-positive breast cancer indicates an endocrine-sensitive but relatively chemotherapy-resistant disease. In contrast, low Tau expression identifies a subset of ER-positive cancers that have poor prognosis with tamoxifen alone and may benefit from adjuvant taxane containing chemotherapy.

These data add to the growing body of evidence that indicate an inverse relationship between endocrine- and chemotherapy-sensitivity in ER-positive breast cancer. It should be noted that in the current paper we present a correlation between a molecular marker and clinical outcome and not the performance characteristics of a new test. In order to validate Tau mRNA expression as a diagnostic predictive test, the measurement methodology, normalization procedure and cut off values to assign positive and negative status will need to be defined a priori. Based on our results, we suggest that if Tau mRNA expression is measured by an Affymetrix U133A gene chip and is normalized according to our normalization procedure (see methods at http://bioinformatics.mdanderson.org/pubdata.html, last visited on Sep. 1, 2006) then Tau levels >183 (lowest tertile) indicate a 78% 10-year distant metastasis-free survival with adjuvant tamoxifen and predict a <4% pCR rate from T/FAC chemotherapy. Tau mRNA levels <183 indicate a 65% 10-year distant metastasis-free survival with adjuvant tamoxifen and predict a 26% pCR rate. However, the true predictive values of Tau mRNA expression levels will need to be determined in an independent study (Simon, 2005). It is also important to consider that inclusion of other genes in addition to Tau in a multigene prediction score can improve its endocrine and chemotherapy response predictive values (Symmans, 2005; Hess 2006).

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A method for assessing the responsiveness and resistance of a tumor to therapy comprising:
(a) obtaining a sample of a tumor from a cancer patient;
(b) evaluating the sample for expression of one or more markers identified in Table 1; and
(c) assessing the responsiveness and resistance of the tumor to therapy based on the evaluation of marker expression in the sample.

2. The method of claim 1, wherein the marker is the gene sequence encoding microtubule-associated Tau.

3. The method of claim 1 wherein the tumor is classified as sensitive to chemotherapy achieves an outcome such as complete pathologic response that is associated with high likelihood of survival.

4. The method of claim 1, wherein the tumor is classified as unlikely to achieve high survival related outcome such as complete pathological response to chemotherapy.

5. The method of claim 1, wherein the therapy is a single chemotherapy agent or a combination of chemotherapy agents such as T/FAC.

6. The method of claim 1, wherein the therapy is an endocrine agent or a combination of endocrine agents.

7. The method of claim 1, wherein the tumor is classified as unlikely to achieve high survival related outcome in response to the endocrine agent described in claim 6.

8. The method of claim 1, wherein the tumor is classified as likely to achieve high-survival related outcome in response to the endocrine agent described in claim 6.

9. The method of claim 1, wherein the tumor comprises breast cancer.

10. The method of claim 1, wherein the sample is obtained by aspiration, biopsy, or surgical resection.

11. The method of claim 1, wherein assessing the expression of the one or more markers comprises detecting mRNA of the one or more markers.

12. The method of claim 11, wherein the detection comprises microarray analysis.

13. The method of claim 11, wherein the detection comprises PCR.

14. The method of claim 11, wherein the detection comprises in situ hybridization.

15. The method of claim 1, wherein assessing the expression of the one or more markers comprises detecting the protein encoded by one or more markers.

16. The method of claim 15, wherein detecting the protein is by immunohistochemistry.

17. The method of claim 2, wherein assessing the expression of gene sequence encoding microtubule-associated Tau comprises detecting mRNA.

18. The method of claim 17, wherein the detection comprises PCR.

19. The method of claim 17, wherein the detection comprises in situ hybridization.

20. The method of claim 2, wherein assessing the expression of gene sequence encoding Tau comprises detecting a microtubule-associated Tau protein.

21. The method of claim 20, wherein detecting the protein is by immunohistochemistry.

22. A method of monitoring a cancer patient receiving chemotherapy or therapy with an endocrine agent comprising:
(a) obtaining a tumor sample from the patient during chemotherapy or therapy with an endocrine agent;
(b) evaluating expression of one or more markers of Table 1 in the tumor sample; and
(c) assessing the cancer patient's responsiveness or resistance to the therapy based on the evaluation of marker expression in the sample.

23. The method of claim 22, further comprising repeating steps (a) to (c) at various time points during the therapy.

24. The method of claim 22, wherein the marker is a microtubule-associated protein Tau marker.

25. A method of assessing anti-cancer activity of a candidate substance comprising:
(a) contacting a first cancer cell with the candidate substance;
(b) comparing expression of one or more markers of Table 1 in the first cancer cell with expression of the markers in a second cancer cell not contacted with the candidate substance; and
(c) assessing the anti-cancer activity of the candidate substance based on the evaluation of marker expression in the first cancer cell and the second cancer cell.

26. The method of claim 25, wherein the anti-cancer activity is sensitization of a cancer cell to therapy.

27. The method of claim 25, wherein the marker is a microtubule-associated protein Tau marker.

28. The method of claim 26, wherein the therapy is a chemotherapy such as T/FAC therapy.

29. The method of claim 26, wherein the therapy is an endocrine agent.

30. A method for diagnosing or staging or monitoring cancer, the method comprising determining the level of one or more markers identified in Table 1 expressed by a cancer cell present in a tissue suspected of being positive for cancer and comparing that level to the level of one or more markers identified in Table 1 expressed by normal tissue, whereby a change in the level in the suspect tissue over the level in the normal tissue indicates responsiveness and resistance of the cancer to therapy.

31. A method for screening a compound that inhibits or prevents cancer cell proliferation, the method comprising determining a first amount one or more markers identified in Table 1 expressed by cancer cells exposed to the compound, wherein the cancer cells have a increased or decreased expression of the one or more markers; and comparing the first amount to a second amount of one or more markers expressed by the cancer cells that have not been exposed to the compound; whereby a difference between the first amount and the second amount indicates that the compound may inhibit or prevent cancer cell proliferation.
